(19) United States
(12) Patent Application Publication
(10) Pub. No.: US 2006/0287315 A1
(43) Pub. Date: Dec. 21, 2006

(54) Pyrazinedicarboxamides and their use

(75) Inventors: Susanne Rohrig, Essen (DE); Mario Jeske, Solingen (DE); Metin Akbaba, Ratingen (DE); Ulrich Rosentreter, Wuppertal (DE); Stephen Boyer, Bethany, CT (US); Karin Fischer, Solingen (DE); Jens Pohlmann, Basel (CH); Arounarith Tuch, Lyon (FR); Elisabeth Perzborn, Wuppertal (DE); Christoph Gerdes, Leverkusen (DE); Karl-Heinz Schlemmer, Wuppertal (DE); Nils Burkhardt, Velbert (DE); Swen Allerheiligen, Essen (DE); Peter Nell, Wuppertal (DE); Sabine Arndt, Dortmund (DE); Mario Lobell, Wuppertal (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 11/299,342

(22) Filed: Dec. 8, 2005

(30) Foreign Application Priority Data
Dec. 9, 2004 (DE) 102004059219.5

(51) Int. Cl. A61K 31/5377; A61K 31/497; C07D 413/02; C07D 403/02

(52) U.S. Cl. 514/235.5; 514/255.05; 544/96; 544/120; 544/405

(57) ABSTRACT

The present invention relates to novel pyridinedicarboxamides, to processes for their preparation, to their use for the treatment and/or prophylaxis of diseases and also to their use for preparing medicaments for the treatment and/or prophylaxis of diseases, in particular thromboembolic disorders.

The present invention relates to novel pyridinedicarboxamides, to processes for their preparation, to their use for the treatment and/or prophylaxis of diseases and also to their use for preparing medicaments for the treatment and/or prophylaxis of diseases, in particular thromboembolic disorders.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a joint reaction path, are distinguished. Here factor Xa, which is formed from the proenzyme factor X, plays a key role, since it connects the two coagulation paths. The activated serine protease Xa cleaves prothrombin to thrombin. The resulting thrombin, in turn, cleaves fibrinogen to fibrin. Subsequent crosslinking of the fibrin monomers causes formation of blood clots and thus haemostasis. In addition, thrombin is a potent effector of platelet aggregation which likewise contributes significantly to haemostasis.

Haemostasis is subject to a complex regulatory mechanism. Uncontrolled activation of the coagulant system or defective inhibition of the activation processes may cause formation of local thrombi or embolisms in vessels (arteries, veins, lymph vessels) or in heart cavities. This may lead to serious thromboembolic disorders. In addition, in the case of consumption coagulopathy, hypercoagulability may—systemically—result in disseminated intravascular coagulation. Thromboembolic complications furthermore occur in microangiopathic haemolytic anaemias, extracorporeal blood circulation, such as haemodialysis, and also in connection with prosthetic heart valves.

Thromboembolic disorders are the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants, i.e. substances for inhibiting or preventing blood coagulation, which are known from the prior art, have various, often grave disadvantages. Accordingly, in practice, an efficient treatment method or prophylaxis of thromboembolic disorders is very difficult and unsatisfactory.

In the therapy and prophylaxis of thromboembolic disorders, use is firstly made of heparin, which is administered parenterally or subcutaneously. Owing to more favourable pharmacokinetic properties, preference is nowadays more and more given to low-molecular-weight heparin; however, even with low-molecular-weight heparin, it is not possible to avoid the known disadvantages described below, which are involved in heparin therapy. Thus, heparin is ineffective when administered orally and has a relatively short half-life. Since heparin inhibits a plurality of factors of the blood coagulation cascade at the same time, the action is nonselective. Moreover, there is a high risk of bleeding; in particular, brain haemorrhages and gastrointestinal bleeding may occur, which may result in thrombopenia, drug-induced alopecia or osteoporosis [Pschyrembel, Klinisches Wörterbuch, 257th edition, 1994, Walter de Gruyter Verlag, page 610, entry “Heparin”; Römpp Lexikon Chemie, Version 1.5, 1998, Georg Thieme Verlag Stuttgart, entry “Heparin”].

A second class of anticoagulants are the vitamin K antagonists. These include, for example, 1,3-indanediones, and especially compounds such as warfarin, phenprocoumon, dicumarol and other coumarin derivatives which inhibit the synthesis of various products of certain vitamin K-dependent coagulation factors in the liver in a non-selective manner. Owing to the mechanism of action, however, the onset of the action is very slow (latency to the onset of action 36 to 48 hours). It is possible to administer the compounds orally; however, owing to the high risk of bleeding and the narrow therapeutic index, a time-consuming individual adjustment and monitoring of the patent are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683].

Recently, a novel therapeutic approach for the treatment and prophylaxis of thromboembolic disorders has been described. This novel therapeutic approach aims to inhibit factor Xa. Because of the central role which factor Xa plays in the blood coagulation cascade, factor Xa is one of the most important targets for anticoagulants [J. Hauptmann, J. Stürzebecher, Thrombosis Research 1999, 93, 203; S. A. V. Raghavan, M. Dikshit, “Recent advances in the status and targets of antithrombotic agents” Drugs Fut. 2002, 27, 669-683; H. A. Wieland, V. Laux, D. Kozian, M. Lorenz, “Approaches in anticoagulation: Rationales for target positioning” Curr. Opin. Investig. Drugs 2003, 4, 264-271; U. J. Ries, W. Wienen, “Serine proteases as targets for antithrombotic therapy” Drugs Fut. 2003, 28, 355-370; L.-A. Linkins, J. I. Weitz, “New anticoagulant therapy” Annu. Rev. Med. 2005, 56, 63-77 (online publication August 2004)].

It has been shown that, in animal models, various both peptidic and nonpeptidic compounds are effective as factor Xa inhibitors. A large number of direct factor Xa inhibitors is already known [J. M. Walenga, W. P. Jeske, D. Hoppensteadt, J. Fareed, “Factor Xa Inhibitors: Today and beyond” Curr. Opin. Investig Drugs 2003, 4, 272-281; J. Ruef, H. A. Katus, “New antithrombotic drugs on the horizon” Expert Opin. Investig. Drugs 2003, 12, 781-797; M. L. Quan, J. M. Smallheer, “The race to an orally active Factor Xa inhibitor: Recent advances” Curr. Opin. Drug Discovery & Development 2004, 7, 460-469]. Nonpeptidic low-molecular-weight factor Xa inhibitors are also described, for example, in WO 03/026652, WO 02/079145, WO 01/019788 and WO 01/064642.

It is an object of the present invention to provide novel substances for controlling disorders, in particular thromboembolic disorders.

The present invention provides compounds of the general formula (I) 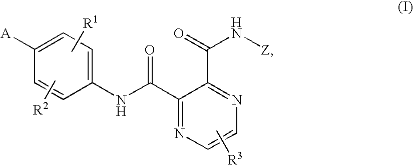
in which
A represents a group of the formula 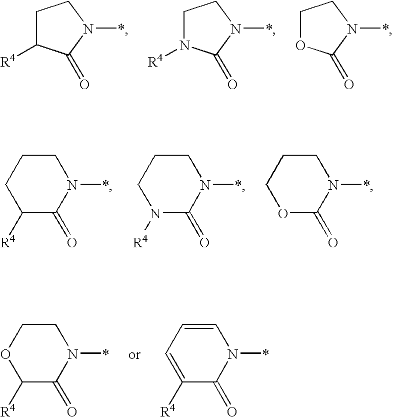
in which
R⁴ represents hydrogen, (C₁-C₆)-alkyl, hydroxyl, (C₁-C₆)-alkoxy, amino, mono- or di-(C₁-C₆)-alkylamino, (C₃-C₇)-cycloalkylamino, (C₁-C₆)-alkanoylamino or (C₁-C₆)-alkoxycarbonylamino, where
(C₁-C₆)-alkyl, (C₁-C₆)-alkoxy, mono- and di-(C₁-C₆)-alkylamino for their part may in each case be substituted by hydroxyl, (C₁-C₄)-alkoxy, amino, mono- or di-(C₁-C₄)-alkylamino, (C₃-C₇)-cycloalkylamino or a 4- to 7-membered saturated heterocycle which is attached via a nitrogen atom and which may contain a ring member from the group consisting of N—R⁵ and O, in which
R⁵ represents hydrogen or (C₁-C₄)-alkyl,
and * represents the point of attachment to the phenyl ring,
z represents phenyl, pyridyl, pyrimidinyl, pyrazinyl or thienyl which may in each case be mono- or disubstituted by identical or different substituents selected from the group consisting of fluorine, chlorine, cyano, (C₁-C₄)-alkyl (which for its part may be substituted by amino) ethynyl and amino,
R¹ and R² are identical or different and independently of one another represent hydrogen, fluorine, chlorine, cyano, (C₁-C₃)-alkyl, cyclopropyl, trifluoromethyl, hydroxyl, (C₁-C₃)-alkoxy, trifluoromethoxy or amino, where
   (C₁-C₃)-alkyl and (C₁-C₃)-alkoxy for their part may be substituted by hydroxyl or amino, and
R³ represents hydrogen or (C₁-C₆)-alkyl, which may be substituted by hydroxyl, (C₁-C₄)-alkoxy, amino or mono- or di-(C₁-C₄)-alkylamino,
   and salts, solvates and solvates of the salts thereof.

Compounds according to the invention are the compounds of the formula (I) and their salts, solvates and solvates of the salts, the compounds, comprised by formula (I), of the formulae mentioned below and their salts, solvates and solvates of the salts and the compounds, comprised by the formula (I), mentioned below as embodiments and their salts, solvates and solvates of the salts if the compounds, comprised by formula (I), mentioned below are not already salts, solvates and solvates of the salts.

Depending on their structure, the compounds according to the invention can exist in stereoisomeric forms (enantiomers, diastereomers). Accordingly, the invention comprises the enantiomers or diastereomers and their respective mixtures. From such mixtures of enantiomers and/or diastereomers, it is possible to isolate the stereoisomerically uniform components in a known manner.

If the compounds according to the invention can be present in tautomeric forms, the present invention comprises all tautomeric forms.

In the context of the present invention, preferred salts are physiologically acceptable salts of the compounds according to the invention. The invention also comprises salts which for their part are not suitable for pharmaceutical applications, but which can be used, for example, for isolating or purifying the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the compounds according to the invention which, in solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the compounds according to the invention. The term “prodrugs” includes compounds which for their part may be biologically active or inactive but which, during the time they spend in the body, are converted into compounds according to the invention (for example metabolically or hydrolytically).

In the context of the present invention, unless specified differently, the substituents have the following meanings:

In the context of the invention, (C₁-C₆)-alkyl, (C₁-C₄)-alkyl and (C₁-C₃)-alkyl represents a straight-chain or branched alkyl radical having 1 to 6, 1 to 4 and 1 to 3 carbon atoms, respectively. Preference is given to a straight-chain or branched alkyl radical having 1 to 4 or 1 to 3 carbon atoms. Particular preference is given to a straight-chain or branched alkyl radical having 1 to 3 carbon atoms. The following radicals may be mentioned by way of example and by way of preference: methyl, ethyl, n-propyl, isopropyl, n-butyl, isobutyl, sec-butyl, tert-butyl, 1-ethylpropyl, n-pentyl and n-hexyl.

In the context of the invention, (C₃-C₇)-cycloalkyl represents a monocyclic cycloalkyl group having 3 to 7 carbon atoms. Preference is given to a cycloalkyl radical having 3 to 6 carbon atoms. The following radicals may be mentioned by way of example and by way of preference: cyclopropyl, cyclobutyl, cyclopentyl, cyclohexyl and cycloheptyl.

In the context of the invention, (C₁-C₆)-alkoxy, (C₁-C₄)-alkoxy and (C₁-C₃)-alkoxy represent a straight-chain or branched alkoxy radical having 1 to 6, 1 to 4 and 1 to 3 carbon atoms, respectively. Preference is given to a straight-chain or branched alkoxy radical having 1 to 4 or 1 to 3 carbon atoms. Particular preference is given to a straight-chain or branched alkoxy radical having 1 to 3 carbon atoms. The following radicals may be mentioned by way of example and by way of preference: methoxy, ethoxy, n-propoxy, isopropoxy, n-butoxy and tert-butoxy.

In the context of the invention, (C₁-C₆)-alkanoyl [(C₁-C₆)-acyl] represents a straight-chain or branched alkyl radical having 1 to 6 carbon atoms which carries a doubly attached oxygen atom in the 1-position and is attached via the 1-position. Preference is given to a straight-chain or branched alkanoyl radical having 1 to 4 carbon atoms. The following radicals may be mentioned by way of example and by way of preference: formyl, acetyl, propionyl, n-butyryl, isobutyryl and pivaloyl.

In the context of the invention, (C₁-C₆)-alkoxycarbonyl represents a straight-chain or branched alkoxy radical having 1 to 6 carbon atoms which is attached via a carbonyl group. Preference is given to a straight-chain or branched alkoxycarbonyl radical having 1 to 4 carbon atoms in the alkoxy group. The following radicals may be mentioned by way of example and by way of preference: methoxycarbonyl, ethoxycarbonyl, n-propoxycarbonyl, isopropoxycarbonyl and tert-butoxycarbonyl.

In the context of the invention, mono-(C₁-C₆)-alkylamino and mono-(C₁-C₄)-alkylamino represent an amino group having a straight-chain or branched alkyl substituent having 1 to 6 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino and tert-butylamino.

In the context of the invention, di-(C₁-C₆)-alkylamino and di-(C₁-C₄)-alkylamino represent an amino group having two identical or different straight-chain or branched alkyl substituents having in each case 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

In the context of the invention, (C₃-C₇)-cycloalkylamino represents an amino group having a cycloalkyl substituent which has 3 to 7 carbon atoms. Preference is given to a cycloalkylamino radical having 3 to 6 carbon atoms. The following radicals may be mentioned by way of example and by way of preference: cyclopropylamino, cyclobutylamino, cyclopentylamino, cyclohexyl-amino and cycloheptylamino.

In the context of the invention, (C₁-C₆)-alkanoylamino represents an amino group having a straight-chain or branched alkanoyl substituent which has 1 to 6 carbon atoms and is attached via the carbonyl group. Preference is given to an alkanoylamino radical having 1 to 4 carbon atoms.

The following radicals may be mentioned by way of example and by way of preference: formamido, acetamido, propionamido, n-butyramido and pivaloylamido.

In the context of the invention, (C₁-C₆)-alkoxycarbonylamino represents an amino group having a straight-chain or branched alkoxycarbonyl substituent which has 1 to 6 carbon atoms in the alkoxy radical and is attached via the carbonyl group. Preference is given to an alkoxycarbonylamino radical having 1 to 4 carbon atoms in the alkoxy group. The following radicals may be mentioned by way of example and by way of preference: methoxycarbonylamino, ethoxycarbonylamino, n-propoxycarbonylamino and tert-butoxycarbonylamino.

In the context of the invention, a 4- to 7-membered heterocycle represents a saturated heterocycle having 4 to 7 ring atoms which contains a ring nitrogen atom and is attached via this ring nitrogen atom and which may contain a further heteroatom from the group consisting of N and O. Preference is given to a 5- or 6-membered saturated heterocycle which is attached via nitrogen and may contain a further heteroatom from the group consisting of N and O. The following radicals may be mentioned by way of example: pyrrolidinyl, oxazolidinyl, imidazolidinyl, piperidinyl, piperazinyl, morpholinyl, azepinyl and 1,4-diazepinyl. Particular preference is given to pyrrolidinyl, piperidinyl, piperazinyl and morpholinyl.

If radicals in the compounds according to the invention are substituted, the radicals can, unless specified otherwise, be mono- or polysubstituted. In the context of the present invention, the meanings of radicals which occur more than once are independent of one another. Substitution with one, two or three identical or different substituents is preferred. Very particular preference is given to substitution with one substituent.

Preference is given to compounds of the formula (I), in which
A represents a group of the formula 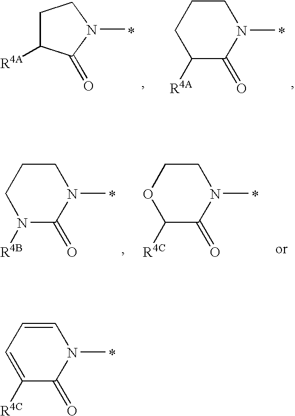
in which
R^{4A} represents hydrogen, hydroxyl, methoxy or amino,
R^{4B} represents methyl or ethyl, each of which may be substituted by hydroxyl, amino, pyrrolidino or cyclopropylamino, or amino,
R^{4C} represents hydrogen, methyl or ethyl, where methyl or ethyl may in each case be substituted by hydroxyl, amino, pyrrolidino or cyclopropylamino,
and
* represents the point of attachment to the phenyl ring,
   z represents a group of the formula 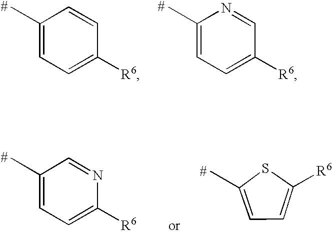
in which
R⁶ represents fluorine, chlorine, methyl, cyano or ethynyl and
# represents the point of attachment to the nitrogen atom,
   R¹ represents hydrogen,
   R² represents hydrogen, fluorine or methyl,
   and
   R³ represents hydrogen,
   and salts, solvates and solvates of the salts thereof.

Particular preference is given to compounds of the formula (I) in which
A represents a heterocyclic group of the formula 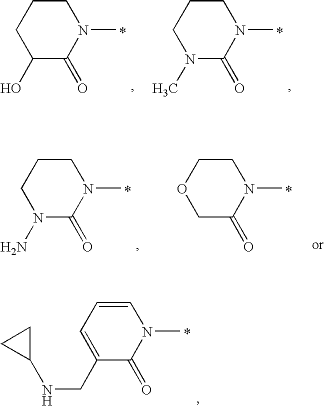
in which * represents the point of attachment to the phenyl ring,
   Z represents a group of the formula 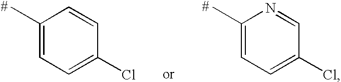
in which # represents the point of attachment to the nitrogen atom,
   R¹ represents hydrogen,
   R² represents hydrogen, fluorine or methyl,
   and
   R³ represents hydrogen,
   and salts, solvates and solvates of the salts thereof.

The individual radical definitions given in the respective combinations or preferred combinations of radicals may, independently of the particular given combination of radicals, also be replaced by any radical definitions of other combinations.

Very particular preference is given to combinations of two or more of the preferred ranges mentioned above.

The invention furthermore provides a process for preparing the compounds accoding to the invention, characterized in that either
[A] compounds of the formula (II) 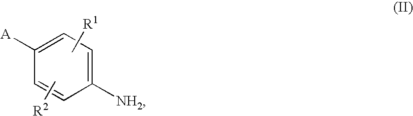
in which A, R¹ and R² are as defined above
are initially, in an inert solvent in the presence of a base, such as, for example, triethylamine, and dehydrating agent, such as, for example, pivaloyl chloride, reacted with a compound of the formula (III) 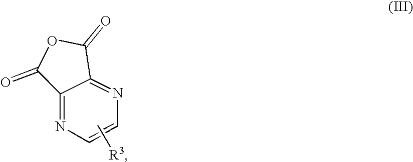
in which R³ is as defined above
to give compounds of the formula (IV) 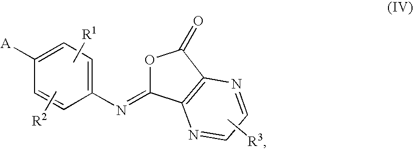
in which A, R¹, R² and R³ are as defined above
and these are then, in an inert solvent in the presence of an acid, converted with a compound of the formula (V)
   H₂N-Z (V),
in which Z is as defined above
into compounds of the formula (I)
   or
   [B] compounds of the formula (V) are initially, in an inert solvent, if appropriate in the presence of a base, reacted with a compound of the formula (III) to give compounds of the formula (VI) 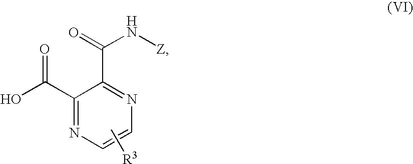
in which R³ and Z are as defined above
and these are then, in an inert solvent after activation of the carboxylic acid function, converted with a compound of the formula (II) into compounds of the formula (I),
   and the compounds of the formula (I) are, if appropriate, converted with the appropriate (i) solvents and/or (ii) bases or acids into their solvates, salts and/or solvates of the salts.

If appropriate, the compounds according to the invention can also be prepared by further conversions of functional groups of individual substituents, in particular the substituents listed under R³ and R⁴, starting with the compounds of the formula (I) obtained by the above process. These conversions are carried out by customary methods and include, for example, reactions such as alkylation, amination, acylation, esterification, ester cleavage, amide formation, oxidation or reduction and also the introduction and removal of protective groups.

Inert solvents for process steps (II)+(III)→(IV) and (IV)+(V)→(I) are, for example, halogenated hydrocarbons, such as dichloromethane, trichloromethane, carbon tetrachloride, 1,2-dichloroethane, trichloroethylene or chlorobenzene, or solvents such as dimethyl sulphoxide, dimethylformamide, N,N′-dimethylpropyleneurea (DMPU), N-methylpyrrolidone (NMP) or acetonitrile. It is also possible to use mixtures of the solvents mentioned. Preference is given to dimethylformamide.

Suitable bases for the process step (II)+(III)→(IV) and, if appropriate, also for the process step (V)+(III)→(VI) are the customary organic amine bases. These include, in particular, triethylamine, N-methylmorpholine, N-methylpiperidine, N,N-diisopropylethylamine, pyridine, 1,5-diazabicyclo[4.3.0]non-5-ene (DBN), 1,4-diazabicyclo[2.2.2]octane (DABCO®) or 1,8-diaza-bicyclo[5.4.0]undec-7-ene (DBU). Preference is given to triethylamine.

Suitable dehydrating agents for process step (II)+(III)→(IV) are, for example, organic carbonyl chloride, such as acetyl chloride or pivaloyl chloride, organic sulphonyl chlorides, such as methanesulphonyl chloride, chloroformic esters, such as methyl chloroformate or isobutyl chloroformate, or inorganic acid chlorides or anhydrides, such as phosphorus oxychloride, phosphorus pentachloride, phosphorus trichloride, phosphorus pentoxide or thionyl chloride. Preference is given to using pivaloyl chloride.

Suitable acids for the process step (IV)+(V)→(I) are, for example, organic carboxylic acids or sulphonic acids, such as acetic acid, trifluoroacetic acid, methanesulphonic acid or trifluoromethanesulphonic acid, or inorganic acids, such as hydrogen chloride, hydrogen bromide, sulphuric acid or phosphoric acid. Preference is given to using trifluoroacetic acid.

Suitable inert solvents for the process step (V)+(III)→(VI) are, for example, halogenated hydrocarbons, such as dichloromethane, trichloromethane, carbon tetrachloride, trichloroethane, tetrachloroethane, 1,2-dichloroethane or trichloroethylene, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as ethyl acetate, acetone, dimethylformamide, dimethyl sulphoxide, N,N′-dimethylpropyleneurea (DMPU), N-methylpyrrolidone (NMP) or acetonitrile. It is also possible to use mixtures of the solvents mentioned. Preference is given to tetrahydrofuran or dimethylformamide.

The process steps (II)+(III)→(IV) and (V)+(III)→(VI) are generally carried out in a temperature range of from −20° C. to +60° C., preferably from 0° C. to +40° C. The reactions can be carried out at atmospheric, elevated or reduced pressure (for example from 0.5 to 5 bar). In general, the reactions are carried out at atmospheric pressure.

The process step (IV)+(V)→(I) is generally carried out in a temperature range of from +20° C. to +100° C., preferably from +50° C. to +80° C. The reaction can be carried out at atmospheric, elevated or reduced presssure (for example from 0.5 to 5 bar). In general, the reaction is carried out at atmospheric pressure.

Inert solvents for the process step (VI)+(H)→(I) are, for example, ethers, such as diethyl ether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethylene or chlorobenzene, or other solvents, such as ethyl acetate, pyridine, dimethyl sulphoxide, dimethylformamide, N,N′-dimethylpropyleneurea (DMPU), N-methylpyrrolidone (NMP), acetonitrile or acetone. It is also possible to use mixtures of the solvents mentioned. Preference is given to dichloromethane, tetrahydrofuran, dimethylformamide or mixtures of these solvents.

Suitable condensing agents for the amide formation in process step (VI)+(II)→(I) are, for example, carbodiimides, such as N,N′-diethyl-, N,N′-dipropyl-, N,N′-diisopropyl-, N,N′-dicyclo-hexylcarbodiimide (DCC), N-(3-dimethylaminoisopropyl)-N′-ethylcarbodiimide hydrochloride (EDC), or phosgene derivatives, such as N,N′-carbonyldiimidazole, or 1,2-oxazolium compounds, such as 2-ethyl-5-phenyl-1,2-oxazolium 3-sulphate or 2-tert-butyl-5-methylisoxazolium perchlorate, or acylamino compounds, such as 2-ethoxy-1-ethoxycarbonyl-1,2-dihydroquinoline, or isobutyl chloroformate, propanephosphonic anhydride (PPA), diethyl cyanophosphonate, bis(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N′N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′N′-tetramethyluronium hexafluorophosphate (HATU) or O-(1H-6-chlorobenzotriazol-1-yl)-1,1,3,3-tetramethyluronium tetrafluoroborate (TCTU), if appropriate in combination with further auxiliaries, such as 1-hydroxybenzotriazole (HOBt) or N-hydroxysuccinimide (HOSu), and also as bases alkali metal carbonates, for example sodium carbonate or potassium carbonate or sodium bicarbonate or potassium bicarbonate, or organic bases, such as trialkylamines, for example triethylamine, N-methylmorpholine, N-methylpiperidine or N,N-diisopropylethylamine. Preference is given to using TBTU, HATU or PPA, in each case in combination with N,N-diisopropyl-ethylamine.

The process step (VI)+(II)→(I) is generally carried out in a temperature range of from −20° C. to +60° C., preferably from 0° C. to +40° C. The reaction can be carried out at atmospheric, elevated or reduced pressure (for example from 0.5 to 5 bar). In general, the reaction is carried out at atmospheric pressure.

The compounds of the formulae (II), (III) and (V) are commercially available, known from the literature, or they can be prepared analogously to processes known from the literature.

The preparation of the compounds according to the invention can be illustrated by the synthesis scheme below:

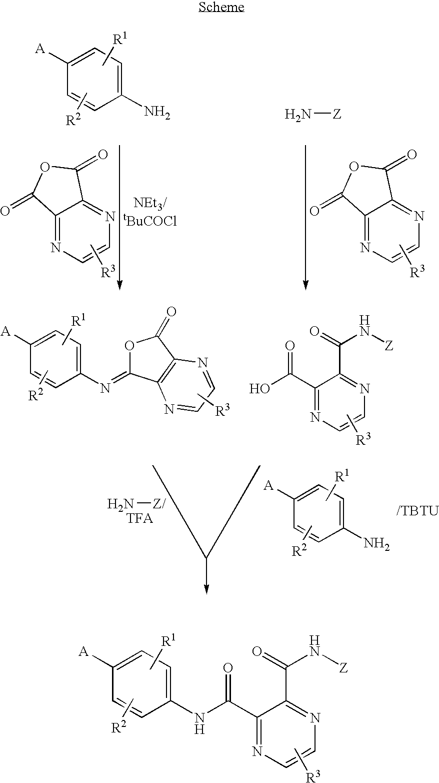

[Abbreviations: ^{t}Bu=tert-butyl; Et=ethyl; TBTU═O-(benzotriazol-1-yl)-N-N—N′,N′-tetramethyl-uronium tetrafluoroborate; TFA=trifluoroacetic acid].

The compounds according to the invention have an unforeseeable useful pharmacological activity spectrum, in particular high efficacy and a favourable half-life.

Accordingly, they are suitable for use as medicaments for the treatment and/or prophylaxis of diseases in humans and animals.

The compounds according to the invention are selective inhibitors of blood coagulation factor Xa which act in particular as anticoagulants.

The present invention furthermore provides the use of the compounds according to the invention for the treatment and/or prophylaxis of disorders, preferably thromboembolic disorders and/or thromboembolic complications.

For the purposes of the present invention, “thromboembolic disorders” include in particular disorders such as ST-elevation mycardial infarction (STEMI) or non-ST-elevation mycardial infarction (non-STEMI), stable angina pectoris, unstable angina pectoris, reocclusions and restenoses after coronary interventions such as angioplasty or aortocoronary bypass, peripheral areterial occlusive diseases, pulmonary embolisms, deep vein thromboses and kidney vein thromboses, transitory ischaemic attacks and also thrombotic and thromboembolic stroke.

Accordingly, the substances are also suitable for preventing and treating cardiogenic thrombo-embolisms, such as, for example, brain ischaemias, stroke and systemic thromboembolisms and ischaemias, in patients having acute, intermittent or persistent cardioarrhythmias, such as, for example, atrial fibrillation, and those undergoing cardioversion, furthermore patients having heart valve disorders or having artificial heart valves. In addition, the compounds according to the invention are suitable for treating disseminated intravascular coagulation (DIC).

Thromboembolic complications furthermore occur during microangiopathic haemolytic anaemias, extracorporal blood circulation, such as haemodialysis, and in connection with heart valve prostheses.

Moreover, the compounds according to the invention are also suitable for the prophylaxis and/or treatment of atherosclerotic vascular disorders and inflammatory disorders, such as rheumatic disorders of the locomotor apparatus, and in addition also for the prophylaxis and/or treatment of Alzheimer's disease. Moreover, the compounds according to the invention can be used for inhibiting tumour growth and formation of metastases, for microangiopathies, age-related macular degeneration, diabetic retinopathy, diabetic nephropathy and other microvascular disorders, and also for the prevention and treatment of thromboembolic complications, such as, for example, venous thromboembolisms, in tumour patients, in particular patients undergoing major surgical interventions or chemo- or radiotherapy.

The compounds according to the invention can additionally also be used for preventing coagulation ex vivo, for example for preserving blood and plasma products, for cleaning/pretreating catheters and other medical tools and instruments, for coating synthetic surfaces of medical tools and instruments used in vivo or ex vivo or for biological samples comprising factor Xa.

The present invention furthermore provides the use of the compounds according to the invention for the treatment and/or prophylaxis of disorders, in particular the disorders mentioned above.

The present invention furthermore provides the use of the compounds according to the invention for preparing a medicament for the treatment and/or prophylaxis of disorders, in particular the disorders mentioned above.

The present invention furthermore provides a method for the treatment and/or prophylaxis of disorders, in particular the disorders mentioned above, using an anticoagulatory effective amount of the compound according to the invention.

The present invention furthermore provides a method for preventing blood coagulation in vitro, in particular in banked blood or biological samples comprising factor Xa, which method is characterized in that an anticoagulatory effective amount of the compound according to the invention is added.

The present invention furthermore provides medicaments comprising a compound according to the invention and one or more further active compounds, in particular for the treatment and/or prophylaxis of the disorders mentioned above. The following compounds may be mentioned by way of example and by way of preference as active compounds suitable for combinations:
lipid-lowering agents, in particular HMG-CoA (3-hydroxy-3-methylglutaryl-coenzyme A) reductase inhibitors;
coronary therapeutics/vasodilators, in particular ACE (angiotensin converting enzyme) inhibitors; AII (angiotensin II) receptor antagonists; β-adrenoceptor antagonists; alpha-1-adrenoceptor antagonists; diuretics; calcium channel blockers; substances which cause an increase in the cyclic guanosine monophosphate (cGMP) concentration such as, for example, stimulators of soluble guanylate cyclase;
plasminogen activators (thrombolytics/fibrinolytics) and compounds enhancing thrombolysis/fibrinolysis, such as inhibitors of the plasminogen activator inhibitor (PAI inhibitors) or inhibitors of the thrombin-activated fibrinolysis inhibitor (TAFI inhibitors);
anticoagulants;
platelet aggregation inhibiting substances (platelet aggregation inhibitors, thrombocyte aggregation inhibitors);
fibrinogen receptor antagonists (glycoprotein-IIb/IIIa antagonists);
and also antiarrhythmics.

The present invention furthermore provides medicaments comprising at least one compound according to the invention, usually together with one or more inert nontoxic pharmaceutically acceptable auxiliaries, and their use for the purposes mentioned above.

The compounds according to the invention can act systemically and/or locally. For this purpose, they can be administered in a suitable way, such as, for example, by the oral, parenteral, pulmonary, nasal, sublingual, lingual, buccal, rectal, dermal, transdermal, conjunctival or otic route, or as implant or stent.

For these administration routes, it is possible to administer the compounds according to the invention in suitable administration forms.

Suitable for oral administration are administration forms which work as described in the prior art and deliver the compounds according to the invention rapidly and/or in modified form, which comprise the compounds according to the invention in crystalline and/or amorphous and/or dissolved form, such as, for example, tablets (uncoated and coated tablets, for example tablets provided with enteric coatings or coatings whose dissolution is delayed or which are insoluble and which control the release of the compound according to the invention), tablets which rapidly decompose in the oral cavity, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (for example intravenously, intraarterially, intracardially, intraspinally or intralumbarly) or with inclusion of absorption (for example intramuscularly, subcutaneously, intracutaneously, percutaneously or intraperitoneally). Administration forms suitable for parenteral administration are, inter alia, preparations for injection and infusion in the form of solutions, suspensions, emulsions, lyophilizates or sterile powders.

Examples suitable for other administration routes are pharmaceutical forms for inhalation (inter alia powder inhalers, nebulizers), nasal drops/solutions/sprays; tablets to be administered lingually, sublingually or buccally, films/wafers or capsules, suppositories, preparations for the eyes or ears, vaginal capsules, aqueous suspensions (lotions, shaking mixtures), lipophilic suspensions, ointments, creams, transdermal therapeutic systems, (e.g. patches), milk, pastes, foams, dusting powders, implants or stents. Preference is given to oral or parenteral administration, in particular oral administration.

The compounds according to the invention can be converted into the stated administration forms.

This can take place in a manner known per se by mixing with inert, nontoxic, pharmaceutically suitable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides) and flavour- and/or odour-masking agents.

In general, it has proved advantageous to administer on parenteral administration amounts of from about 0.001 to 1 mg/kg, preferably from about 0.01 to 0.5 mg/kg, of body weight to achieve effective results. The dosage on oral administration is from about 0.01 to 100 mg/kg, preferably about 0.01 to 20 mg/kg, and very particularly preferably 0.1 to 10 mg/kg, of body weight.

It may nevertheless be necessary, where appropriate, to deviate from the amounts mentioned, depending on the body weight, the administration route, the individual response to the active compound, the mode of preparation and the time or interval over which administration takes place. Thus, in some cases it may be sufficient to make do with less than the aforementioned minimal amount, whereas in other cases the upper limit mentioned must be exceeded. In the event of administration of larger amounts, it may be advisable to divide these into a plurality of individual doses over the day.

The invention is illustrated by the working examples below. The invention is not limited to the examples.

The percentage data in the following tests and examples are percentages by weight unless otherwise indicated; parts are parts by weight. Solvent ratios, dilution ratios and concentration data of liquid/liquid solutions are in each case based on volume.

### A. EXAMPLES

Abbreviations and Acronyms:

| | |
|---|---|
| DCI | direct chemical ionization (in MS) |
| DMF | N,N-dimethylformamide |
| DMSO | dimethyl sulphoxide |
| ee | enantiomeric excess |
| eq. | equivalent(s) |
| ESI | electrospray ionization (in MS) |
| h | hour(s) |
| HPLC | high pressure, high performance liquid chromatography |
| LC-MS | liquid chromatography-coupled mass spectroscopy |
| min | minute(s) |
| MS | mass spectroscopy |
| NMR | nuclear magnetic resonance spectroscopy |
| RP | reverse phase (in HPLC) |
| RT | room temperature |
| Rₜ | retention time (in HPLC) |
| THF | tetrahydrofuran |

LC-MS and HPLC Methods:
Method 1:

MS instrument: Micromass ZQ; HPLC instrument: Waters Alliance 2795; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 2:

MS instrument: Micromass ZQ; HPLC instrument: HP 1100 Series; UV DAD; column: Phenomenex Synergi 2μ Hydro-RP Mercury 20 mm×4 mm; mobile phase A: 111 of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 3:

Instrument: Micromass Quattro LCZ with HPLC Agilent Series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 208-400 nm.

Method 4:

Instrument: Micromass Platform LCZ with HPLC Agilent Series 1100; column: Phenomenex Synergi 2μ, Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 5:

Instrument: Micromass Platform LCZ mit HPLC Agilent Series 1100; column: Thermo HyPURITY Aquastar 3μ 50 mm×2.1 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Method 6:

MS instrument: Micromass ZQ; HPLC instrument: Waters Alliance 2795; column: Merck Chromolith SpeedROD RP-18e 50 mm×4.6 mm; mobile phase A: 1 l of water+0.5 ml of 50% strength formic acid, mobile phase B: 1 l of acetonitrile+0.5 ml of 50% strength formic acid; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

Method 7:

Instrument: HP 1100 with DAD detection; column: Kromasil 100 RP-18, 60 mm×2.1 mm, 3.5 μm; mobile phase A: 5 ml of HClO₄ (70%)/1 of water, mobile phase B: acetonitrile; gradient: 0 min 2% B→0.5 min 2% B→4.5 min 90% B→9 min 0% B→9.2 min 2% B→10 min 2% B; flow rate: 0.75 ml/min; column temperature: 30° C.; UV detection: 210 nm.

Method 8:

Instrument: HP 1100 with DAD detection; column: Kromasil 100 RP-18, 60 mm×2.1 mm, 3.5 μm; mobile phase A: 5 ml of HClO₄ (70%)/l of water, mobile phase B: acetonitrile; gradient: 0 min 2% B→0.5 min 2% B→4.5 min 90% B→15 min 90% B→15.2 min 2% B→16 min 2% B; flow rate: 0.75 ml/min; column temperature: 30° C.; UV detection: 210 nm.

Method 9:

Instrument: HP 1100 with DAD detection; column: Kromasil 100 RP-18, 60 mm×2.1 mm, 3.5 μm; mobile phase A: 5 ml of HClO₄ (70%)/l of water, mobile phase B: acetonitrile; gradient: 0 min 2% B→0.5 min 2% B 4.5 min 90% B→6.5 min 90% B→6.7 min 2% B→7.5 min 2% B; flow rate: 0.75 ml/min; column temperature: 30° C.; UV detection: 210 nm.

Starting Materials and Intermediates:

### Example 1A

### 1-(4-Aminophenyl)pyrrolidin-2-one

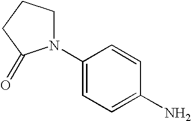

The compound was prepared by reduction of 1-(4-nitrophenyl)-2-pyrrolidinone, see Reppe et al., Justus Liebigs Ann. Chem. 1955, 596, 209.

### Example 2A

### 3-(4-Aminophenyl)-1,3-oxazolidin-2-one

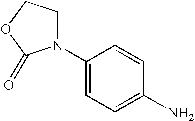

The compound was prepared by a route known from the literature, see M. Artico et al., Farmaco Ed. Sci. 1969, 24, 179-190.

### Example 3A

### 1-(4-Aminophenyl)imidazolidin-2-one

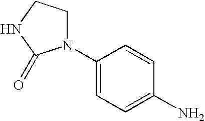

2.0 g (9.6 mmol) of 1-(4-nitrophenyl)imidazolidin-2-one [prepared by Mitsunobu reaction of 1-(2-hydroxyethyl)-3-(4-nitrophenyl)urea, see T. H. Kim, G. J. Lee, M.-H. Cha, Synth. Commun. 1999, 29, 2753-2758] are dissolved in 20 ml of DMF/THF (1:1), 200 mg of palladium-on-carbon (5%) are added and the mixture is hydrogenated at RT and atmospheric pressure in an atmosphere of hydrogen. After 12 h, the reaction mixture is, using Tonsil, filtered off with suction through Celite, the filter cake is washed with THF, the filtrate is concentrated and the residue is dried under high vacuum.

Yield: 1.7 g (93% of theory)

LC-MS (method 7): Rₜ=0.31 min;

MS (ESIpos): m/z=178 [M+H]⁺.

### Example 4A

### 1-(4-Amino-3-fluorophenyl)-3-hydroxypiperidin-2-one

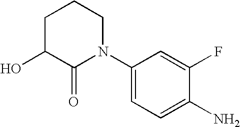

The compound is prepared analogously to a method known from the literature [A. Klapers et al., J. Am. Chem. Soc. 2002, 124, 7421-7428] from 2-fluoro-4-iodoaniline and 3-hydroxypiperidin-2-one [preparation see I. S. Hutchinson et al., Tetrahedron 2002, 58, 3137-3143]:

A suspension of 6.45 g (27.2 mmol) of 2-fluoro-4-iodoaniline, 3.92 g (34.0 mmol, 1.25 eq.) of 3-hydroxypiperidin-2-one, 1.04 g (5.5 mmol, 0.2 eq.) of copper(I) iodide, 11.56 g (54.5 mmol, 2 eq.) of potassium phosphate and 1.2 ml (10.9 mmol, 0.4 eq.) of N,N-dimethylethylenediamine in 157 ml of dioxane is, under argon, stirred under reflux overnight. A further 1.04 g (5.5 mmol, 0.2 eq.) of copper(I) iodide and 0.9 ml (8.2 mmol, 0.3 eq.) of N,N-dimethylethylenediamine are added, and the reaction mixture is stirred under reflux for another 8 h. The suspension is filtered through a layer of kieselguhr and the residue is washed with a mixture of dichloromethane and methanol (1:1). The combined filtrates are concentrated under reduced pressure. The crude product is purified by flash chromatography (silica gel 60, mobile phase: dichloromethane/methanol 100:1→40:1).

Yield: 2.57 g (41% of theory)

HPLC (method 9): Rₜ=1.52 min;

MS (DCI, NH₃): m/z=242 [M+NH₄]⁺;

¹H-NMR (300 MHz, DMSO-d₆): δ=6.94 (d, 1H), 6.81-6.65 (m, 2H), 5.12 (br. s, 2H), 3.99 (dt, 1H), 3.63-3.39 (m, 2H), 2.12-2.00 (m, 1H), 2.00-1.62 (m, 4H).

### Example 5A

### tert-Butyl[1-(4-aminophenyl)-2-oxopiperidin-3-yl]carbamate

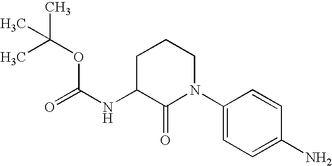

The compound is prepared analogously to a method known from the literature [A. Klapers et al., J. Am. Chem. Soc. 2002, 124, 7421-7428] from 7.48 g (34.2 mmol) of 4-iodoaniline and 9.00 g (42.0 mmol, 1.23 eq.) of tert-butyl(2-oxopiperidin-3-yl)carbamate [preparation see K.-L. Yu et al., J. Med. Chem. 1988, 31, 1430-1436]; see also Example 4A.

Yield: 6.4 g (60% of theory)

HPLC (method 9): Rₜ=3.50 min;

MS (ESIpos): m/z=306 [M+H]⁺;

¹H-NMR (300 MHz, DMSO-d₆): δ=6.85 (d, 1H), 6.52 (d, 2H), 5.00 (s, 2H), 4.10-3.94 (m, 1H), 3.52-3.42 (m, 2H), 2.08-1.95 (m, 1H), 1.95-1.69 (m, 3H).

### Example 6A

### 4-(4-Aminophenyl)morpholin-3-one

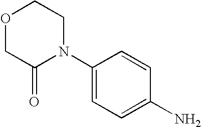

The compound is prepared by substitution of 4-fluoronitrobenzene with morpholin-3-one [J.-M. Lehn, F. Montavon, Helv. Chim. Acta 1976, 59, 1566-1583] and subsequent reduction of the 4-(4-nitrophenyl)morpholin-3-one (see WO 01/47919, starting materials I and II, pp. 55-57).

### Example 7A

### 4-(4-Amino-3-fluorophenyl)morpholin-3-one

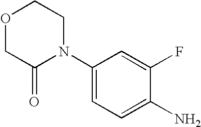

The compound was prepared analogously to a method known from the literature [A. Klapers et al., J. Am. Chem. Soc. 2002, 124, 7421-7428] from 5.0 g (21.1 mmol) of 2-fluoro-4-iodaniline and 2.6 g (26 mmol, 1.23 eq.) of morpholin-3-one; see also Example 4A.

Yield: 4.2 g (94% of theory)

HPLC (method 2): Rₜ=0.85 min;

MS (ESIpos): m/z=211 [M+H]⁺;

¹H-NMR (300 MHz, DMSO-d₆): δ=7.06 (dd, 1H), 6.87 (dd, 1H), 6.73 (dd, 1H), 5.18 (br. s, 2H), 4.14 (s, 2H), 3.92 (t, 2H), 3.62 (t, 2H).

### Example 8A

### 4-(4-Aminophenyl)-2-(2-{[tert-butyl(dimethyl)silyl]oxy}ethyl)morpholin-3-one

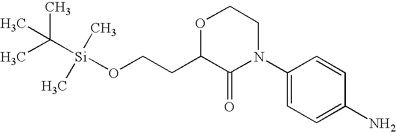

### Step a): 2-Allyl-4-(4-nitrophenyl)morpholin-3-one

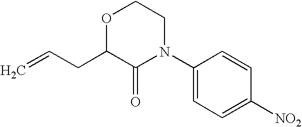

At −70° C. and under argon, 13.0 g (58.5 mmol) of 4-(4-nitrophenyl)morpholin-3-one [J.-M. Lehn, F. Montavon, Helv. Chim. Acta 1976, 59, 1566-1583] are added to a solution of 13.7 g (81.9 mmol, 1.4 eq.) of lithium 1,1,1,3,3,3-hexamethyldisilazan-2-ide in 400 ml THF. The reaction mixture is stirred for 10 min, and 5.4 ml (58.5 mmol, 1.0 eq.) of 3-iodo-1-propene which had been filtered with THF through a little alumina beforehand are then added. The reaction mixture is allowed to slowly warm to RT and concentrated to a volume of about 100 ml, and a mixture of dichloromethane and water is added. After phase separation, the aqueous phase is extracted with dichloromethane and the organic phases are dried over magnesium sulphate, filtered and concentrated under reduced pressure. The crude product is purified by flash chromatography (silica gel 60, mobile phase: cyclohexane→cyclohexane/ethyl acetate 1:1).

Yield: 4.1 g (27% of theory)

LC-MS (method 1): Rₜ=1.78 min;

MS (ESIpos): m/z=263 [M+H]⁺.

### Step b): 2-(2-Hydroxyethyl)-4-(4-nitrophenyl)morpholin-3-one

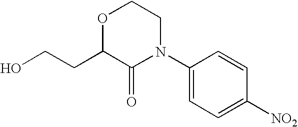

At RT, 0.62 ml (0.10 mmol, 0.02 eq.) of a 4% strength aqueous osmium tetroxide solution and 3.25 g (15.2 mmol, 3 eq.) of sodium periodate are added to a solution of 1.33 g (5.07 mmol) of 2-allyl-4-(4-nitrophenyl)morpholin-3-one in 60 ml of tetrahydrofuran/water (1:1). The reaction mixture is stirred at RT for 1.3 h and diluted with a mixture of water and dichloromethane. After phase separation, the aqueous phase is extracted with dichloromethane, and the combined organic phases are dried over magnesium sulphate, filtered and concentrated under reduced pressure. The residue is taken up in 40 ml of tetrahydrofuran/water (1:1), at RT, 96 mg (2.5 mmol, 0.5 eq.) of sodium borohydride are added to the reaction solution and the mixture is stirred at RT for 5 min. After addition of a mixture of water and dichloromethane and phase separation, the aqueous phase is extracted with dichloromethane, and the combined organic phases are dried over sodium sulphate, filtered and concentrated under reduced pressure. The crude product is purified by glash chromatography (silica gel 60, mobile phase: dichloromethane→dichloromethane/methanol 10:1).

Yield: 1.1 g (80% of theory)

LC-MS (method 2): Rₜ=1.49 min;

MS (ESIpos): m/z=267 [M+H]⁺.

### Step c): 2-(2-{[tert-Butyl(dimethyl)silyl]oxy}ethyl)-4-(4-nitrophenyl)morpholin-3-one

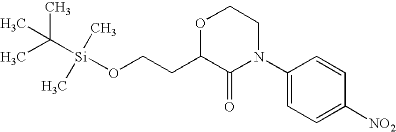

At RT, 563 mg (8.26 mmol, 2 eq.) of imidazole and 934 mg (6.20 mmol, 1.5 eq.) of tert-butyldimethylsilyl chloride are added to a solution of 1.10 g (4.13 mmol) of 2-(2-hydroxyethyl)-4-(4-nitrophenyl)morpholin-3-one in 7 ml of DMF, and the mixture is stirred at 90° C. overnight. The reaction mixture is added to a saturated aqueous sodium bicarbonate solution. After addition of diethyl ether and phase separation, the aqueous phase is extracted with diethyl ether, and the combined organic phases are washed with saturated aqueous sodium chloride solution, dried over magnesium sulphate, filtered and concentrated under reduced pressure. The crude product is used for the next step without further purification.

Yield: 1.67 g (purity 97%)

LC-MS (method 1): Rₜ=2.87 min;

MS (ESIpos): m/z=381 [M+H]⁺.

### Step d): 4-(4-Aminophenyl)-2-(2-{[tert-butyl(dimethyl)silyl]oxy}ethyl)morpholin-3-one

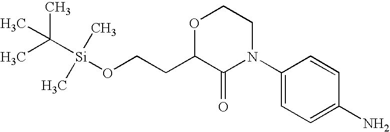

At RT, 5.0 ml of a 5% strength aqueous iron trichloride solution and 652 mg (11.7 mmol, 3.7 eq.) of iron powder are added to a solution of 1.2 g (3.15 mmol) of 2-(2-{[tert-butyl(dimethyl)silyl]oxy}ethyl)-4-(4-nitrophenyl)morpholin-3-one in 15 ml of ethanol and 10 ml of water and the mixture is stirred under reflux for 2.5 h. The hot reaction solution is filtered through Celite and concentrated under reduced pressure. After addition of water and dichloromethane, the reaction soluton is made alkaline using a drop of ammonia solution and filtered again through Celite. After phase separation, the aqueous phase is extracted with dichloromethane, and the combined organic phases are dried over magnesium sulphate, filtered and concentrated under reduced pressure.

Yield: 899 mg (purity 95%, 77% of theory)

LC-MS (method 1): Rₜ=2.23 min;

MS (ESIpos): m/z=351 [M+H]⁺,

¹H-NMR (300 MHz, CDCl₃): δ=7.00 (d, 2H), 6.61 (d, 2H), 4.30 (dd, 1H), 4.08-3.98 (m, 1H), 3.87 (dd, 1H), 3.82-3.70 (m, 3H), 3.50-3.39 (m, 1H), 2.36-2.21 (m, 1H), 1.95-1.81 (m, 1H), 0.84 (s, 9H), 0.01 (s, 6H).

### Example 9A

### 1-(4-Aminophenyl)-3-methyltetrahydropyrimidin-2(1H)-one

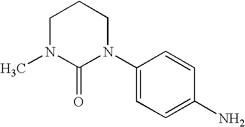

### Step a): 1-Methyl-3-(4-nitrophenyl)tetrahydropyrimidin-2(1H)-one

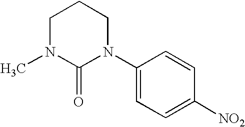

Under argon and at RT, 14.8 g (131.4 mmol, 1.5 eq.) of potassium tert-butoxide are added to a solution of 10.0 g (87.6 mmol) of 1-methyltetrahydropyrimidin-2(1H)-one [preparation see DE 1 121 617; Chem. Abstr. 1962, 56, 11601 g] in 300 ml of DMF, and the mixture is stirred at RT for 45 min. A little at a time, 14.8 g (105.1 mmol, 1.2 eq.) of 1-fluoro-4-nitrobenzene are then added to the reaction mixture, the mixture is stirred at RT overnight and then concentrated under reduced pressure. The residue is taken up in a mixture of saturated aqueous sodium bicarbonate solution and ethyl acetate. After phase separation, the aqueous phase is extracted with ethyl acetate. The combined organic phases are washed with saturated aqueous sodium chloride solution, dried over sodium sulphate, filtered and concentrated under reduced pressure. The residue is stirred in toluene, filtered off and dried under reduced pressure.

Yield: 9.4 g (46% of theory)

LC-MS (method 2): Rₜ=1.68 min;

MS (ESIpos): m/z=236 [M+H]⁺;

¹H-NMR (300 MHz, DMSO-d₆): δ=8.14 (d, 2H), 7.55 (d, 2H), 3.77 (t, 2H), 3.38 (t, 2H), 2.90 (s, 3H), 2.05 (t, 2H).

### Step b): 1-(4-Aminophenyl)-3-methyltetrahydropyrimidin-2(1H)-one

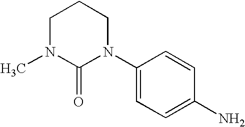

Under argon, 2.0 g of palladium-on-carbon (5%) are added to a solution of 10.0 g (42.5 mmol) of 1-methyl-3-(4-nitrophenyl)tetrahydropyrimidin-2(1H)-one in 400 ml THF, and the mixture is hydrogenated in an atmosphere of hydrogen at RT and atmospheric pressure overnight. With Tonsil, the reaction mixture is filtered off through Celite, the filter cake is washed with methanol and the filtrate is concentrated under reduced pressure and dried.

Yield: 8.6 g (99% of theory)

LC-MS (method 5): Rₜ=2.00 min;

MS (ESIpos): m/z=206 [M+H]⁺,

¹H-NMR (300 MHz, DMSO-d₆): δ=6.82 (d, 2H), 6.48 (d, 2H), 4.88 (br. s, 2H), 3.49 (t, 2H), 3.28 (t, 2H), 2.80 (s, 3H), 1.98 (quintet, 2H).

### Example 10A

### 1-(4-Aminophenyl)-3-(2-hydroxyethyl)tetrahydropyrimidin-2(1H)-one

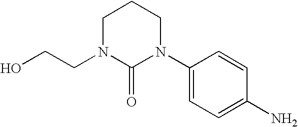

### Step a): 1-(2-Hydroxyethyl)-3-(4-nitrophenyl)tetrahydropyrimidin-2(1H)-one

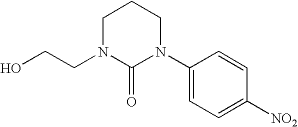

The compound is prepared analogously to a method known from the literature [A. Klapers et al., J. Am. Chem. Soc. 2002, 124, 7421-7428] from 5.00 g (20.1 mmol) of 1-iodo-4-nitrobenzene and 3.56 g (24.7 mmol, 1.23 eq.) of 1-(2-hydroxyethyl)tetrahydropyrimidin-2(1H)-one [preparation see DE 1 121 617; Chem. Abstr. 1962, 56, 11601g]; see also Example 4A.

Yield: 2.93 g (51% of theory)

LC-MS (method 2): Rₜ=1.49 min;

MS (ESIpos): m/z=266 [M+H]⁺.

### Step b): 1-(4-Aminophenyl)-3-(2-hydroxyethyl)tetrahydropyrimidin-2(1H)-one

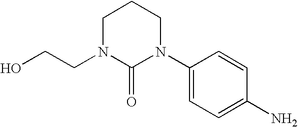

Under argon, 35 mg of palladium-on-carbon (10%) are added to a solution of 200 mg (0.75 mmol) of 1-(2-hydroxyethyl)-3-(4-nitrophenyl)tetrahydropyrimidin-2(1H)-one in 10 ml of methanol, and the mixture is hydrogenated in an atmosphere of hydrogen at RT and atmospheric pressure for 2 h. The catalyst is removed through a layer of silica gel and the filtrate is concentrated under reduced pressure. The residue is stirred in diethyl ether, filtered off and dried under reduced pressure.

Yield: 156 mg (88% of theory)

HPLC (method 9): Rₜ=2.47 min;

MS (ESIpos): m/z=236 [M+H]⁺;

¹H-NMR (300 MHz, DMSO-d₆): δ=6.84 (d, 2H), 6.48 (d, 2H), 4.92 (s, 1H), 3.54-3.43 (m, 4H), 3.39 (t, 2H), 3.29 (t, 2H), 1.95 (q, 2H).

### Example 11A

### 1-(4-Amino-3-fluorophenyl)-3-(hydroxymethyl)pyridin-2(1H)-one

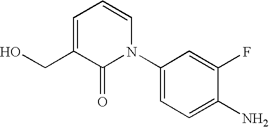

The compound is prepared analogously to the synthesis of Example 4A from 5.39 g (22.8 mmol) of 4-iodo-2-fluoroaniline and 4.00 g (28.5 mmol, 1.25 eq.) of 3-(hydroxymethyl)pyridin-2(1H)-one [preparation see S. McN. Sieburth et al., Chem. Commun. 1996, 19, 2249-2250].

Yield: 3.46 g (65% of theory)

HPLC (method 9): Rₜ=2.44 min;

MS (ESIpos): m/z=235 [M+H]⁺;

¹H-NMR (300 MHz, DMSO-d₆): δ=7.48 (m, 2H), 7.10 (dd, 1H), 6.89 (dd, 1H), 6.80 (t, 1H), 6.30 (t, 1H), 5.39 (s, 2H), 5.11 (t, 1H), 4.31 (d, 2H).

### Example 12A

### 3-{[(5-Chloropyridin-2-yl)amino]carbonyl}pyrazine-2-carboxylic acid

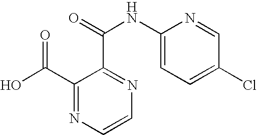 68.0 g (0.53 mol) of 2-amino-5-chloropyridine are dissolved in 1100 ml of THF, and 95.3 g (0.63 mol) of 2,3-pyrazinedicarboxylic anhydride are added a little at a time. The suspension is stirred at room temperature for one hour. The precipitate is then filtered off. The filtrate is concentrated and the residue is combined with the precipitate. The product is stirred in diethyl ether, filtered again and dried under reduced pressure.

Yield: 154 g (99% of theory)

HPLC (method 9): Rₜ=3.50 min;

MS (ESIpos): m/z=279 [M+H]⁺;

¹H-NMR (300 MHz, DMSO-d₆): δ=13.89 (br. s, 1H), 11.07 (s, 1H), 8.90 (dd, 2H), 8.44 (s, 1H), 8.20 (d, 1H), 8.01 (dd, 1H).

### Example 13A

### 3-{[(4-Ethynylphenyl)amino]carbonyl}pyrazine-2-carboxylic acid

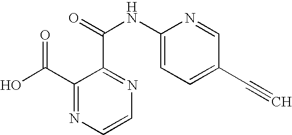

500 mg (4.27 mmol) of 4-ethynylaniline and 641 mg (4.27 mmol) of 2,3-pyrazinedicarboxylic anhydride are reacted analogously to the method described for Example 12A.

Yield: 1.08 g (purity 96%, 91% of theory)

HPLC (method 3): Rₜ=1.49 min;

MS (ESIpos): m/z=224 [M+H—CO₂]⁺.

### Example 14A

### 3-{[(4-Chlorophenyl)amino]carbonyl}pyrazine-2-carboxylic acid

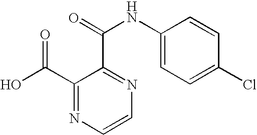

100 mg (0.78 mmol) of 4-chloroaniline and 118 mg (0.78 mmol) of 2,3-pyrazinedicarboxylic anhydride are reacted analogously to the method described for Example 12A.

Yield: 115 mg (53% of theory)

HPLC (method 1): Rₜ=1.28 min;

MS (ESIpos): m/z=234 [M+H—CO₂]⁺;

¹H-NMR (300 MHz, DMSO-d₆): δ=13.80 (br. s, 1H), 10.92 (s, 1H), 8.90 (dd, 2H), 7.82 (d, 2H), 7.44 (d, 2H).

### Example 15A

### 7-{[4-(2-Oxo-1,3-oxazolidin-3-yl)phenyl]imino}furo[3,4-b]pyrazin-5(7H)-one

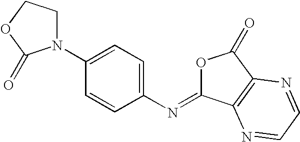

Under argon, 1.07 g (6 mmol) of 3-(4-aminophenyl)-1,3-oxazolidin-2-one (Example 2A) are dissolved in 15 ml of absolute DMF. 0.9 g (6 mmol) of pyrazinedicarboxylic anhydride are then added, and the reaction solution is stirred for 1 h. After addition of 0.75 ml (0.55 g, 5.4 mmol) of triethylamine and 0.81 ml (0.8 g, 6.6 mmol) of pivaloyl chloride, the reaction mixture is stirred for 0.5 h at RT and then diluted with water. The crystals are filtered off with suction, washed with methanol and tert-butyl methyl ether and dried under reduced pressure.

Yield: 1.4 g (75% of theory)

LC-MS (method 3): Rₜ=1.51 min;

MS (ESIpos): m/z=311 [M+H]⁺,

¹H-NMR (300 MHz, DMSO-d₆): δ=9.05 (s, 2H), 7.75 (d, 2H), 7.5 (d, 2H), 4.5 (tr, 2H), 4.15 (tr, 2H).

### Example 16A

### 7-{[4-(2-Oxopyrrolidin-1-yl)phenyl]imino}furo[3,4-b]pyrazin-5(7H)-one

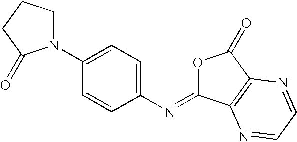

The compound is prepared analogously to the synthesis of Example 15A from 1.06 g (6 mmol) of 1-(4-aminophenyl)pyrrolidin-2-one (Example 1A) and 0.9 g (6 mmol) of pyrazinedicarboxylic anhydride.

Yield: 0.98 g (53% of theory)

LC-MS (method 3): Rₜ=1.59 min;

MS (ESIpos): m/z=309 [M+H]⁺;

¹H-NMR (300 MHz, DMSO-d₆): δ=9.05 (s, 2H), 7.85 (d, 2H), 7.45 (d, 2H), 3.9 (tr, 2H), 2.55 (tr, 2H), 2.1 (quintet, 2H).

### Example 17A

### 7-{[4-(2-Oxoimidazolidin-1-yl)phenyl]imino}furo[3,4-b]pyrazin-5(7H)-one

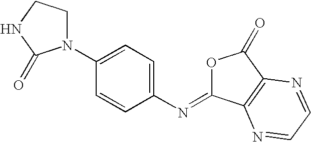

The compound is prepared analogously to the synthesis of Example 15A from 1.32 g (7.45 mmol) of 1-(4-aminophenyl)imidazolidin-2-one (Example 3A) and 1.12 g (7.45 mmol) of pyrazine-dicarboxylic anhydride.

Yield: 1.08 g (47% of theory)

LC-MS (method 1): Rₜ=1.18 min;

MS (ESIpos): m/z=310 [M+H]⁺;

¹H-NMR (300 MHz, DMSO-d₆): δ=9.05 (s, 2H), 7.75 (d, 2H), 7.4 (d, 2H), 7.05 (s, 1H), 3.9 (tr, 2H), 3.45 (tr, 2H).

### Example 18A

### 7-{[4-(3-Oxomorpholin-4-yl)phenyl]imino}furo[3,4-b]pyrazin-5(7H)-one

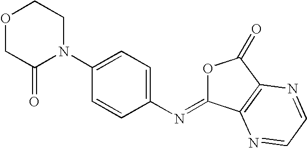

The compound is prepared analogously to the synthesis of Example 15A from 1.15 g (6 mmol) of 4-(4-aminophenyl)morpholin-3-one (Example 6A) and 0.9 g (6 mmol) of pyrazinedicarboxylic anhydride.

Yield: 1.3 g (69% of theory)

LC-MS (method 2): Rₜ=1.30 min;

MS (ESIpos): m/z=325 [M+H]⁺;

¹H-NMR (300 MHz, DMSO-d₆): δ=9.1 (s, 2H), 7.6 (d, 2H), 7.5 (d, 2H), 4.25 (s, 2H), 4.05 (tr, 2H), 3.8 (dd, 2H).

### Example 19A

### 7-{[4-(3-Methyl-2-oxotetrahydropyrimidin-1(2H)-yl)phenyl]imino}furo[3,4-b]pyrazin-5(7H)-one

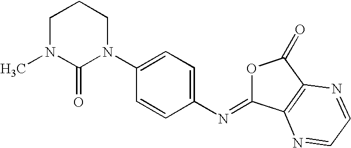

The compound is prepared analogously to the synthesis of Example 15A from 1.23 g (6 mmol) of 1-(4-aminophenyl)-3-methyltetrahydropyrimidin-2(1H)-one (Example 9A) and 0.9 g (6 mmol) of pyrazinedicarboxylic anhydride.

Yield: 1.3 g (64% of theory)

LC-MS (method 3): Rₜ=1.56 min;

MS (ESIpos): m/z=338 [M+H]⁺;

¹H-NMR (300 MHz, DMSO-d₆): δ=9.05 (s, 2H), 7.45 (d, 2H), 7.35 (d, 2H), 3.75 (tr, 2H), 3.4 (tr, 2H), 2.9 (s, 3H), 2.1 (quintet, 2H).

### Example 20A

### N-(2-{[tert-Butyl(dimethyl)silyl]oxy}ethyl)benzene-1,4-diamine

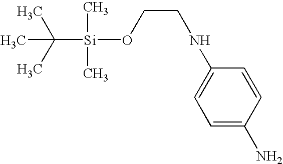

### Step a): 2-[(4-Nitrophenyl)amino]ethanol

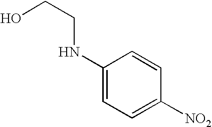

130 ml (2.15 mol, 3 eq.) of 2-aminoethanol and 274 ml (1.57 mol, 2.2 eq.) of N,N-diisopropyl-ethylamine are added to a solution of 101 g (716 mmol) of 4-fluoronitrophenol in 500 ml of ethanol. The reaction mixture is stirred at 50° C. overnight, a further 86 ml (1.43 mol, 2.0 eq.) of 2-aminoethanol and 249 ml (1.43 mol, 2.0 eq.) of N,N-diisopropylethylamine are then added and the mixture is stirred at 50° C. for a further 12 h. The reaction solution is concentrated under reduced pressure and the residue is stirred with 600 ml of water. The precipitate formed is filtered off, washed repeatedly with water and dried.

Yield: 127 g (97% of theory)

LC-MS (method 5): Rₜ=2.32 min;

MS (ESIpos): m/z=183 [M+H]⁺,

¹H-NMR (300 MHz, DMSO-d₆): δ=7.99 (d, 2H), 7.30 (t, 1H), 6.68 (d, 2H), 4.82 (t, 1H), 3.63-3.52 (m, 2H), 3.30-3.19 (m, 2H).

### Step b): N-(2-{[tert-Butyl(dimethyl)silyl]oxy}ethyl)-4-nitroaniline

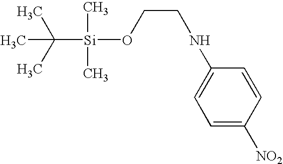

At RT, 30.6 g (203 mmol, 1.2 eq.) of tert-butyldimethylchlorosilane and 17.3 g (254 mmol, 1.5 eq.) of imidazole are added to a solution of 30.8 g (169 mmol) of 2-[(4-nitrophenyl)amino]ethanol in 300 ml of DMF, and the mixture is stirred at RT for 2.5 h. The reaction mixture is concentrated under reduced pressure and the residue is dissolved in 200 ml of dichloromethane and 100 ml of water. After phase separation, the aqueous phase is extracted three times with in each case 80 ml of dichloromethane. The combined organic phases are washed with 100 ml of saturated aqueous sodium chloride solution, dried over sodium sulphate, filtered and concentrated under reduced pressure.

Yield: 49.7 g (quant.)

LC-MS (method 3): Rₜ=3.09 min;

MS (ESIpos): m/z=297 [M+H]⁺;

¹H-NMR (300 MHz, DMSO-d₆): δ=7.98 (d, 2H), 7.29 (t, 1H), 6.68 (d, 2H), 3.77-3.66 (m, 2H), 3.35-3.24 (m, 2H), 0.81 (s, 9H), 0.0 (s, 6H).

### Step c): N-(2-{[tert-Butyl(dimethyl)silyl]oxy}ethyl)benzene-1,4-diamine

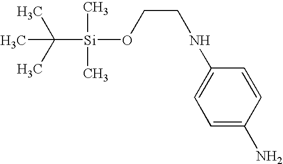

Under argon, 4 g of palladium-on-carbon (10%) are added to a solution of 59.5 g (201 mmol) of N-(2-{[tert-butyl(dimethyl)silyl]oxy}ethyl)-4-nitroaniline in 500 ml of ethanol, and the mixture is hydrogenated in an atmosphere of hydrogen at RT and atmospheric pressure. The catalyst is removed over a filter layer and washed with ethanol, and the filtrate is concentrated under reduced pressure.

Yield: 53 g (quant.)

LC-MS (method 2): Rₜ=1.83 min;

MS (ESIpos): m/z=267 [M+H]⁺;

¹H-NMR (300 MHz, DMSO-d₆): δ=6.42-6.30 (m, 4H), 4.48 (t, 1H), 4.21 (br. s, 2H), 3.68-3.58 (m, 2H), 3.04-2.93 (m, 2H), 0.82 (s, 9H), 0.0 (s, 6H).

### Example 21A

### 3-{[(4-Cyanophenyl)amino]carbonyl}pyrazine-2-carboxylic acid

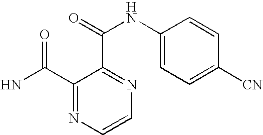

1.0 g (8.5 mmol) of 4-aminobenzonitrile and 1.3 g (8.5 mmol) of 2,3-pyrazinedicarboxylic anhydride are reacted analogously to the method described for Example 12A.

Yield: 2.1 g (92% of theory)

HPLC (method 3): Rₜ=1.06 min;

MS (ESIpos): m/z=225 [M+H—CO₂]⁺;

¹H-NMR (300 MHz, DMSO-d₆): δ=13.92 (br. s, 1H), 11.22 (s, 1H), 8.92 (dd, 2H), 7.99 (d, 2H), 7.87 (d, 2H).

### Example 22A

### 2-{2-[2-Amino-5-(3-oxomorpholin-4-yl)phenoxy]ethyl}-1H-isoindole-1,3(2H)-dione

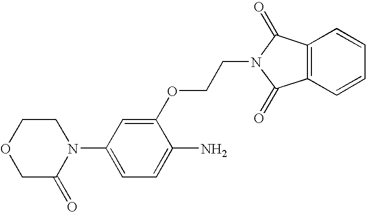

### Step a): 2-[2-(5-Fluoro-2-nitrophenoxy)ethyl]-1H-isoindole-1,3(2H)-dione

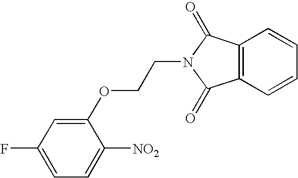

130 ml of DMF and 92.6 g (670.2 mmol, 1.5 eq.) of potassium carbonate are added to 70.2 g (446.8 mmol) of 5-fluoro-2-nitrophenol. A solution of 11.1 g (67.0 mmol, 0.15 eq.) of potassium iodide and 113.5 g (446.8 mmol, 1.0 eq.) of N-(2-bromoethyl)phthalimide in 200 ml of DMF is added. The suspension is stirred at 80° C. for 16 hours. After cooling to room temperature, 1300 ml of water are added. The mixture is stirred at room temperature for one hour and the solid is then filtered off. The residue is washed in each case three times with 200 ml of water and 200 ml of diethyl ether. The solid is dried at 85° C. under high vacuum.

Yield: 89.1 g (purity 90%, 54% of theory)

LC-MS (method 1): Rₜ=2.09 min;

MS (ESIpos): m/z=331 [M+H]⁺,

¹H-NMR (400 MHz, DMSO-d₆): δ=7.97-7.93 (m, 1H), 7.91-7.82 (m, 4H), 7.38 (dd, 1H), 6.97 (dd, 1H), 4.45 (t, 2H), 4.00 (t, 2H).

### Step b): 2-{2-[2-Nitro-5-(3-oxomorpholin-4-yl)phenoxy]ethyl}-1H-isoindole-1,3(2H)-dione

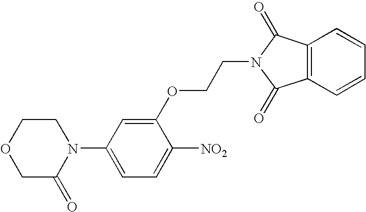

3.02 g (26.87 mmol, 1.5 eq.) of potassium tert-butoxide and 7.10 g (21.50 mmol, 1.2 eq.) of 2-[2-(5-fluoro-2-nitrophenoxy)ethyl]-1H-isoindole-1,3(2H)-dione are added to a solution of 1.81 g (17.91 mmol) of morpholin-3-one [E. Pfeil, U. Harder, Angew. Chem. 1967, 79, 188] in 100 ml of DMF. The mixture is stirred at room temperature for 18 hours and then at 80° C. for 4 hours. After cooling, 400 ml of water and 200 ml of dichloromethane are added. The organic phase is separated off and the aqueous phase is re-extracted twice with in each case 100 ml of dichloromethane. The combined organic phases are concentrated under reduced pressure. The residue is purified by column chromatography on silica gel (mobile phase: dichloromethane/methanol 98:2) and then by preparative RP-HPLC.

Yield: 940 mg (13% of theory)

LC-MS (method 1): Rₜ=1.73 min;

MS (ESIpos): m/z=412 [M+H]⁺,

¹H-NMR (400 MHz, DMSO-d₆): δ=7.91-7.84 (m, 5H), 7.47 (d, 1H), 7.20 (dd, 1H), 4.41 (t, 2H), 4.24 (s, 2H), 4.02-3.97 (m, 4H), 3.81 (t, 2H).

### Step c): 2-{2-[2-Amino-5-(3-oxomorpholin-4-yl)phenoxy]ethyl}-1H-isoindole-1,3(2H)-dione

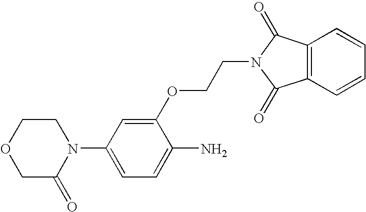

121 mg (0.11 mmol, 0.05 eq.) of palladium-on-carbon (10%) are added to a suspension, covered with argon, of 935 mg (2.27 mmol) of 2-{2-[2-nitro-5-(3-oxomorpholin-4-yl)phenoxy]ethyl}-1H-isoindole-1,3(2H)-dione in 100 ml of an ethanol/methanol/dichloromethane mixture (1:1:1). The mixture is hydrogenated at room temperature and atmospheric pressure for 16 hours. After filtration through kieselguhr, the solution is concentrated under reduced pressure and the residue is purified by column chromatography on silica gel (mobile phase: ethyl acetate/methanol 98:2). The resulting product is stirred in diethyl ether and dried under high vacuum.

Yield: 547 mg (purity 78%, 49% of theory)

LC-MS (method 1): Rₜ=1.37 min;

MS (ESIpos): m/z=382 [M+H]⁺.

Working Examples:

General Method 1: Opening of the phenyliminomino[3,4-b]pyrazin-5(7H)-ones

Under argon, 0.1 mmol of phenyliminofuro[3,4-b]pyrazin-5(7H)-one is dissolved in 0.15 ml of absolute DMF. 7.7 μl (11.4 mg, 0.1 mmol) of trifluoroacetic acid and 0.1 or 0.2 mmol of aniline derivative are then added, and the reaction mixture is stirred at 70° C. overnight. After cooling, the reaction mixture is diluted with 0.1 ml of DMF and a little water and filtered. The filtrate is purified by preparation HPLC (column: Machery Nagel VP50/21 Nucleosil 100-5 C18 Nautilus, 5 μm, 21×50 mm; injection volume: 500 μl; mobile phase A=water+0.1% formic acid, mobile phase B=acetonitrile; gradient: 0 min 10% B→2 min 10% B→6 min 90% B→7 min 90% B→7.1 min 10% B→8 min 10% B; flow rate: 25 ml/min; wavelength: 220 nm). The product-containing fractions are concentrated under reduced pressure.

General Method 2: Amide Coupling—Process I

3-{[Arylamino]carbonyl}pyrazine-2-carboxylic acid and N,N-diisopropylethylamine (1.05 eq.) are initially charged in dichloromethane and stirred at RT for 15 min. A solution of the aniline derivative (1.0 eq.) in dichloromethane is then added dropwise. O-(Benzotriazol-1-yl)-N,N,N′,N′-tetramethyluronium tetrafluoroborate (1.05 eq.) is added, and the mixture is stirred at room temperature overnight. The reaction solution is then washed with water, with saturated aqueous sodium bicarbonate solution and again with water. The solvent is removed under reduced pressure, and ethyl acetate is added to the residue. The precipitated solid is filtered off and washed with pentane. The filtrate is concentrated under reduced pressure and the residue is purified by column chromatography.

General Method 3: Amide Coupling—Process II

N,N-Diisopropylethylamine (10.0 eq.) is added to a solution of the aniline derivative (1.0 eq.) in dichloromethane. 3-{[Arylamino]carbonyl}pyrazine-2-carboxylic acid (1.1 eq.) and n-propane-phosphonic anhydride (n-PPA) (2.0 eq.) are then added. The reaction suspension is stirred at RT overnight. The solvent is removed under reduced pressure. The residue is taken up in DMSO and purified by RP-HPLC (mobile phase: water/acetonitrile 90:10→2:98).

The following compounds are prepared by the General method 1, starting with Example 16A:

### Example 1

### N-(4-Chlorophenyl)-N′-[4-(2-oxopyrrolidin-1-yl)phenyl]pyrazin-2,3-dicarboxamide

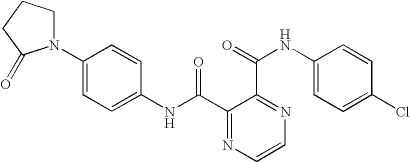

Yield: 11.3 mg (26% of theory)

LC-MS (method 1): Rₜ=1.84 min;

MS (ESIpos): m/z=436 [M+H]⁺.

### Example 2

### N-(5-Chloropyridin-2-yl)-N′-[4-(2-oxopyrrolidin-1-yl)phenyl]pyrazine-2,3-dicarboxamide

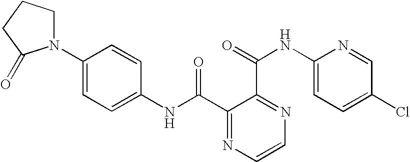

Yield: 3 mg (7% of theory)

LC-MS (method 2): Rₜ=1.96 min;

MS (ESIpos): m/z=437 [M+H]⁺.

### Example 3

### N-(6-Chloropyridin-3-yl)-N′-[4-(2-oxopyrrolidin-1-yl)phenyl]pyrazine-2,3-dicarboxamide

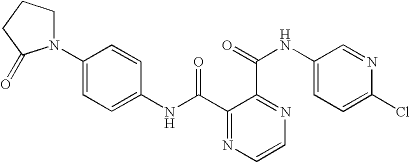

Yield: 12 mg (27% of theory)

LC-MS (method 2): Rₜ=1.76 min;

MS (ESIpos): m/z=437 [M+H]⁺.

### Example 4

### N-(4-Fluorophenyl)-N′-[4-(2-oxopyrrolidin-1-yl)phenyl]pyrazine-2,3-dicarboxarmide

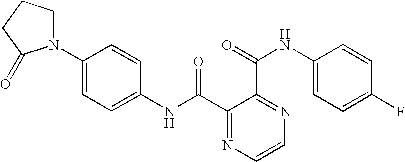

Yield: 26 mg (62% of theory)

LC-MS (method 4): Rₜ=2.07 min;

MS (ESIpos): m/z=420 [M+H]⁺.

### Example 5

### 10 N-(4-Methylphenyl)-N′-[4-(2-oxopyrrolidin-1-yl)phenyl]pyrazine-2,3-dicarboxamide

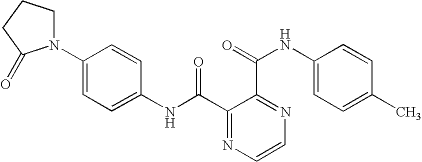

Yield: 14 mg (34% of theory)

LC-MS (method 2): Rₜ=1.98 min;

MS (ESIpos): m/z=416 [M+H]⁺.

### Example 6

### N-(4-Chlorophenyl)-N′-[4-(2-oxo-1,3-oxazolidin-3-yl)phenyl]pyrazine-2,3-dicarboxamide

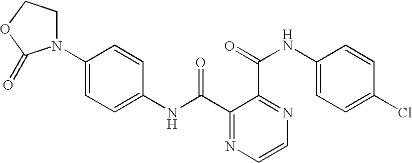

The title compound is prepared according to the General method 1, starting with Example 15A.

Yield: 5 mg (11% of theory)

LC-MS (method 1): Rₜ=1.82 min;

MS (ESIpos): m/z=438 [M+H]⁺.

### Example 7

### N-(5-Chloropyridin-2-yl)-N′[4-(2-oxo-1,3-oxazolidin-3-yl)phenyl]pyrazine-2,3-dicarboxamide

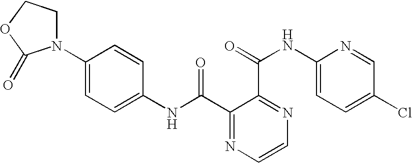

### Step a): N-{4-[(2-{[tert-Butyl(dimethyl)silyl]oxy}ethyl)amino]phenyl}-N′-(5-chloropyridin-2-yl)pyrazine-2,3-dicarboxamide

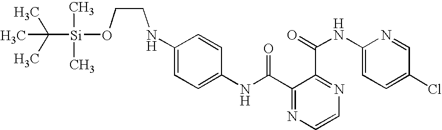

According to General method 2, 104.5 g (0.38 mol) of the compound from Example 12A are reacted with 100.0 g (0.38 mol) of the compound from Example 20A.

Yield: 101.3 g (51% of theory)

LC-MS (method 3): Rₜ=2.96 min;

MS (ESIpos): m/z=527 [M+H]⁺;

¹H-NMR (400 MHz, DMSO-d₆): δ=11.07 (s, 1H), 10.37 (s, 1H), 8.85 (s, 2H), 8.38 (s, 1H), 8.21 (d, 1H), 7.95 (d, 1H), 7.45 (d, 2H), 6.53 (d, 2H), 5.40 (t, NH), 3.67 (t, 2H), 3.10 (dt, 2H), 0.83 (s, 9H), 0.00 (s, 6H).

### Step b): N-(5-Chloropyridin-2-yl)-N′-{4-[(2-hydroxyethyl)amino]phenyl}pyrazine-2,3-dicarboxamide

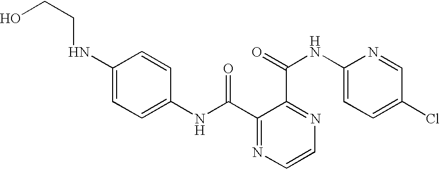

10.0 g (19.0 mmol) of N-{4-[(2-{[tert-butyl(dimethyl)silyl]oxy}ethyl)amino]phenyl}-N′-(5-chloro-pyridin-2-yl)pyrazine-2,3-dicarboxamide are dissolved in 50 ml of THF, and 37.9 ml (9.9 g, 37.9 mmol) of tetra-n-butylammonium fluoride are added at 0° C. The reaction solution is allowed to warm to room temperature and stirred at this temperature for 1 h. The solvent is removed under reduced pressure and dichloromethane and water are added to the residue. The organic phase is removed, dried over sodium sulphate, filtered and concentrated under reduced pressure. The resulting solid is stirred in diethyl ether, filtered off and dried under reduced pressure.

Yield: 4.9 g (63% of theory)

LC-MS (method 2): Rₜ=1.54 min;

MS (ESIpos): m/z=413 [M+H]⁺.

### Step c): N-(5-Chloropyridin-2-yl)-N′-[4-(2-oxo-1,3-oxazolidin-3-yl)phenyl]pyrazine-2,3-dicarboxamide

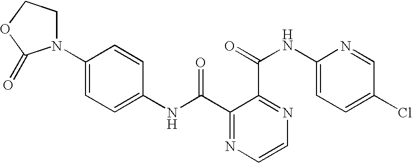

295 mg (1.8 mmol) of 1,1′-carbonyldiimidazole are added to 500 mg (1.2 mmol) of N-(5-chloropyridin-2-yl)-N′-{4-[(2-hydroxyethyl)amino]phenyl}pyrazine-2,3-dicarboxamide in 20 ml of THF. 74 mg (0.6 mmol) of N,N-4-dimethylaminopyridine are then added, and the reaction mixture is stirred at 80° C. for 16 h. The solvent is removed under reduced pressure and the residue is purified by preparative HPLC.

Yield: 100 mg (19% of theory)

HPLC (method 7): Rₜ=3.89 min;

MS (ESIpos): m/z=439 [M+H]⁺;

¹H-NMR (400 MHz, DMSO-d₆): δ=11.12 (s, 1H), 10.80 (s, 1H), 8.96 (s, 2H), 8.42 (s, 1H), 8.24 (d, 1H), 7.99 (d, 1H), 7.81 (d, 2H), 7.53 (dd, 2H), 4.43 (t, 2H), 4.05 (t, 2H).

### Example 8

### N-(4-Chlorophenyl)-N′-[4-(2-oxoimidazolidin-1-yl)phenyl]pyrazine-2,3-dicarboxamide

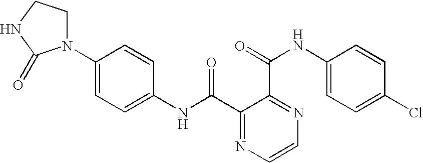

According to the General method 1, the title compound is prepared starting with Example 17A.

Yield: 34.9 mg (40% of theory)

LC-MS (method 3): Rₜ=1.92 min;

MS (ESIpos): m/z=437 [M+H]⁺.

### Example 9

### N-(5-Chloropyridin-2-yl)-N′-[2-fluor-4-(3-hydroxy-2-oxopiperidin-1-yl)phenyl]pyrazine-2,3-di-carboxamide

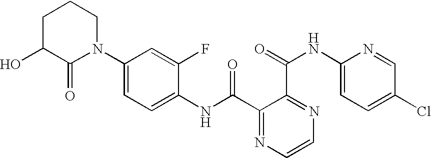

According to the General method 2, 2.57 g (11.46 mol) of the compound from Example 4A are reacted with 3.19 g (11.46 mol) of the compound from Example 12A.

Yield: 3.23 g (58% of theory)

LC-MS (method 2): Rₜ=1.94 min;

MS (ESIpos): m/z=485 [M+H]⁺,

¹H-NMR (400 MHz, DMSO-d₆): δ=11.06 (s, 1H), 10.54 (s, 1H), 8.96 (s, 2H), 8.41 (s, 1H), 8.23 (d, 1H), 7.99 (dd, 1H), 7.86 (t, 1H), 7.41 (dd, 1H), 7.18 (d, 1H), 5.30 (d, 1H), 4.12-4.02 (m, 1H), 3.74-3.64 (m, 1H), 3.61-3.50 (m, 1H), 2.17-2.04 (m, 1H), 2.02-1.81 (m, 2H), 1.81-1.69 (m, 1H).

The enantiomers are separated by chromatography on a chiral phase [column: KBD 5326, 640×40 mm, based on the selector poly(N-methacryloyl-L-leucinedicyclopropylmethylamide); injection volume: 10 ml; mobile phase A: isohexane, mobile phase B: ethyl acetate; gradient: 0 min 70% B→40 min 100% B→45 min 70% B; flow rate: 50 ml/min; column temperature: 24° C.; wavelength: 280 nm].

Enantiomer 1:

>99% ee, Rₜ=3.58 min [column: ent-KBD 5326, 250×4.6 mm; mobile phase: ethyl acetate; flow rate: 1.0 ml/min; column temperature: 24° C.; wavelength: 270 nm].

Enantiomer 2:

98% ee, Rₜ=4.38 min [column: KBD 5326, 250×4.6 mm; mobile phase: ethyl acetate; flow rate: 1.0 ml/min; column temperature: 24° C.; wavelength: 270 nm].

### Example 10

### N-[4-(3-Amino-2-oxopiperidin-1-yl)phenyl]-N′-(4-chlorophenyl)pyrazine-2,3-dicarboxamide hydrochloride

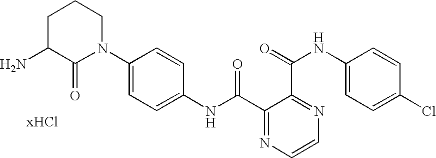

Step a): tert-Butyl[1-(4-{[(3-{[(4-chlorophenyl)amino]carbonyl}pyrazin-2-yl)carbonyl]-amino}phenyl)-2-oxopiperidin-3-yl]carbamate

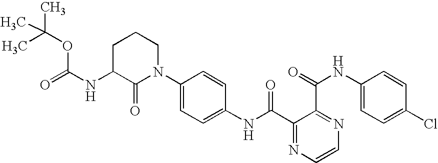

According to the General method 3, 560 mg (1.83 mmol) of the compound from Example 5A are reacted with 560 mg (2.02 mmol) of the compound from Example 14A.

Yield: 0.12 g (12% of theory)

LC-MS (method 1): Rₜ=2.13 min;

MS (ESIpos): m/z=565 [M+H]⁺;

¹H-NMR (400 MHz, DMSO-d₆): δ=10.88 (s, 1H), 10.78 (s, 1H), 8.95 (s, 2H), 7.79 (d, 2H), 7.74 (d, 2H), 7.42 (d, 2H), 7.25 (d, 2H), 6.99 (d, 1H), 4.16-4.03 (m, 1H), 3.69-3.52 (m, 2H), 2.09-1.76 (m, 4H), 1.39 (s, 9H).

### Step b): N-[4-(3-Amino-2-oxopiperidin-1-yl)phenyl]-N′-(4-chlorophenyl)pyrazine-2,3-dicarboxamide hydrochloride

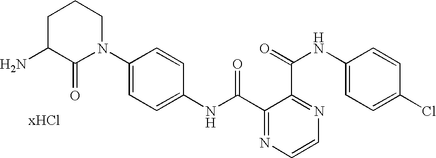

2 ml of a 4 N solution of hydrogen chloride in dioxane are added at RT to a solution of 100 mg (0.18 mmol) of tert-butyl[1-(4-{[(3-{[(4-chlorophenyl)amino]carbonyl}pyrazin-2-yl)carbonyl]-amino}phenyl)-2-oxopiperidin-3-yl]carbamate in 5 ml of dioxane. The reaction suspension is stirred at RT overnight. The solvent is removed under reduced pressure and the residue is stirred in diethyl ether, filtered off with suction and dried under high vacuum.

Yield: 76 mg (92% of theory)

LC-MS (method 1): Rₜ=1.21 min;

MS (ESIpos): m/z=465 [M+H]⁺;

¹H-NMR (400 MHz, DMSO-d₆): δ=10.92 (s, 1H), 10.85 (s, 1H), 8.97 (s, 2H), 8.42-8.28 (m, 3H), 7.8 (t, 4H), 7.41 (d, 2H), 7.3 (d, 2H), 4.13-4.0 (m, 1H), 3.78-3.53 (m, 2H), 2.21-2.31 (m, 1H), 1.98-2.31 (m, 1H), 1.98-1.84 (m, 1H).

According to the General method 1, the following compound is prepared starting with Example 18A:

### Example 11

### N-(4-Chlorophenyl)-N′-[4-(3-oxomorpholin-4-yl)phenyl]pyrazine-2,3-dicarboxamide

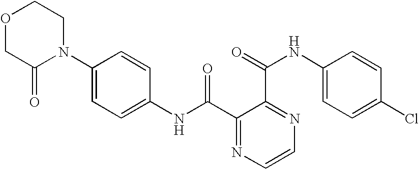

Yield: 9.7 mg (22% of theory)

LC-MS (method 1): Rₜ=1.71 min;

MS (ESIpos): m/z=452 [M+H]⁺;

¹H-NMR (200 MHz, DMSO-d₆): δ=10.9 (s, 1H), 10.83 (s, 1H), 8.97 (s, 2H), 7.9-7.7 (m, 4H), 7.5-7.3 (m, 4H), 4.21 (s, 2H), 3.98 (t, 2H), 3.61 (t, 2H).

### Example 12

### N-(5-Chloropyridin-2-yl)-N′[4-(3-oxomorpholin-4-yl)phenyl]pyrazine-2,3-dicarboxamide

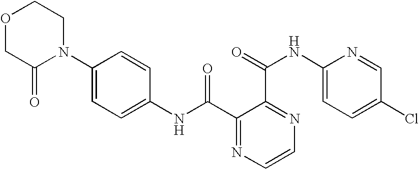

According to the General method 2, 10.0 g (34.1 mmol) of the compound from Example 12A are reacted with 6.6 g (34.1 mmol) of the compound from Example 6A.

Yield: 12.3 g (79% of theory)

LC-MS (method 4): Rₜ=1.84 min;

MS (ESIpos): m/z=453 [M+H]⁺;

¹H-NMR (300 MHz, DMSO-d₆): δ 11.06 (s, 1H), 10.82 (s, 1H), 8.93 (s, 2H), 8.41 (s, 1H), 8.23 (d, 1H), 8.02-7.92 (dd, 1H), 7.8 (d, 2H), 7.36 (d, 2H), 4.19 (s, 2H), 3.97 (t, 2H), 3.71 (t, 2H).

### Example 13

### N-(5-Chloropyridin-2-yl)-N′-[2-fluoro-4-(3-oxomorpholin-4-yl)phenyl]pyrazine-2,3-dicarboxamide

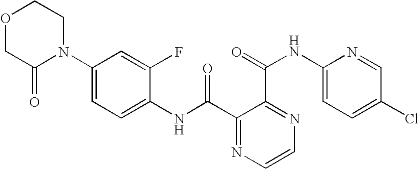

According to the General method 3, 500 mg (1.79 mmol) of the compound from Example 7A are reacted with 415 mg (1.97 mmol) of the compound from Example 12A.

Yield: 96 mg (11% of theory)

LC-MS (method 2): Rₜ=1.90 min;

MS (ESIpos): m/z=471 [M+H]⁺;

¹H-NMR (400 MHz, DMSO-d₆): δ=11.05 (s, 1H), 10.57 (s, 1H), 8.98 (s, 2H), 8.42 (s, 1H), 8.25 (d, 1H), 7.98 (d, 1H), 7.87 (t, 1H), 7.48 (d, 1H), 7.29 (d, 1H), 4.22 (s, 2H), 3.98 (t, 2H), 3.77 (t, 2H).

### Example 14

### N-(6-Chloropyridin-3-yl)-N′-[4-(3-oxomorpholin-4-yl)phenyl]pyrazine-2,3-dicarboxamide

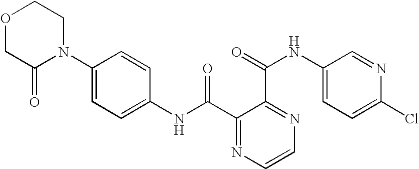

Yield: 5 mg (11% of theory)

LC-MS (method 3): Rₜ=1.6 min;

MS (ESIpos): m/z=453 [M+H]⁺.

### Example 15

### N-(2-Chlorophenyl)-N′-[4-(3-oxomorpholin-4-yl)phenyl]pyrazine-2,3-dicarboxamide

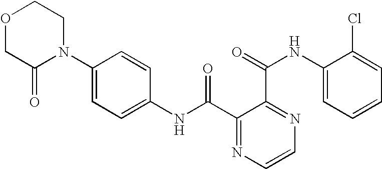

Yield: 0.2 mg (0.2% of theory)

LC-MS (method 3): Rₜ=1.92 min;

MS (ESIpos): m/z=452 [M+H]⁺.

### Example 16

### N-(3,5-Dichlorophenyl)-N′-[4-(3-oxomorpholin-4-yl)phenyl]pyrazine-2,3-dicarboxamide

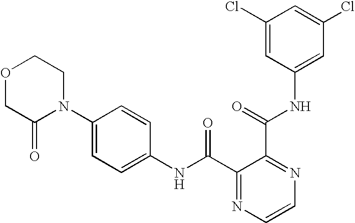

Yield: 6 mg (12% of theory)

LC-MS (method 3): Rₜ=2.13 min;

MS (ESIpos): m/z=486 [M+H]⁺.

### Example 17

### N-(4-Fluorophenyl)-N′[4-(3-oxomorpholin-4-yl)phenyl]pyrazine-2,3-dicarboxamide

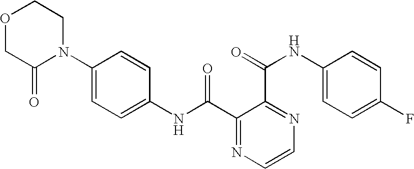

Yield: 28 mg (64% of theory)

LC-MS (method 4): Rₜ=1.95 min;

MS (ESIpos): m/z=436 [M+H]⁺.

### Example 18

### N-(4-Methylphenyl)-N′-[4-(3-oxomorpholin-4-yl)phenyl]pyrazine-2,3-dicarboxamide

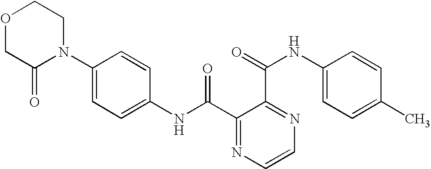

Yield: 24 mg (56% of theory)

LC-MS (method 3): Rₜ=1.82 min;

MS (ESIpos): m/z=432 [M+H]⁺.

### Example 19

### N-[4-(3-Oxomorpholin-4-yl)phenyl]-N′-phenylpyrazine-2,3-dicarboxamide

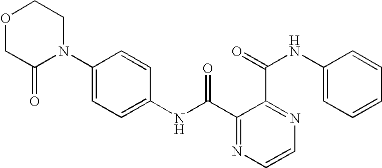

Yield: 31 mg (74% of theory)

LC-MS (method 2): Rₜ=1.65 min;

MS (ESIpos): m/z=418 [M+H]⁺.

### Example 20

### N-(4-Ethynylphenyl)-N′-[4-(3-oxomorpholin-4-yl)phenyl]pyrazine-2,3-dicarboxamide

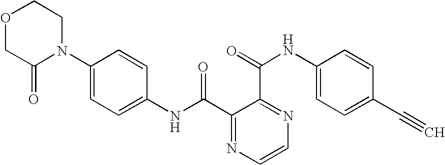

According to the General method 2, 72 mg (0.37 mmol) of the compound from Example 6A are reacted with 100 mg (0.37 mmol) of the compound from Example 13A.

Yield: 19 mg (11% of theory)

LC-MS (method 1): Rₜ=1.61 min;

MS (ESIpos): m/z=442 [M+H]⁺;

¹H-NMR (300 MHz, DMSO-d₆): δ=10.92 (s, 1H), 10.84 (s, 1H), 8.96 (s, 2H), 7.78 (dd, 4H), 7.48 (d, 2H), 7.38 (d, 2H), 4.20 (s, 2H), 4.13 (s, 1H), 3.97 (dd, 2H), 3.72 (dd, 2H).

### Example 21

### N-(4-Chlorophenyl)-N′-{4-[2-(2-hydroxyethyl)-3-oxomorpholin-4-yl]phenyl}pyrazine-2,3-dicarboxamide

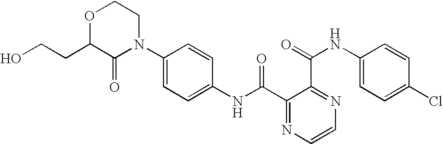

### Step a): N-{4-[2-(2-{[tert-Butyl(dimethyl)silyl]oxy}ethyl)-3-oxomorpholin-4-yl]phenyl}-N′-(4-chlorophenyl)pyrazine-2,3-dicarboxamide

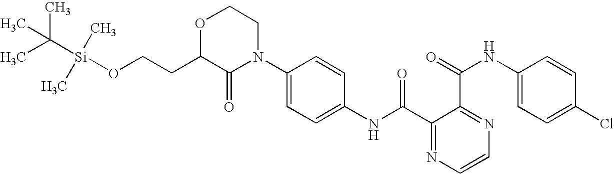

At RT and under argon, 64 mg (0.43 mmol, 1.0 eq.) of 2,3-pyrazinedicarboxylic anhydride are added to a solution of 150 mg (0.43 mmol) of 4-(4-aminophenyl)-2-(2-{[tert-butyl(dimethyl)silyl]-oxy}ethyl)morpholin-3-one (Example 8A) in 1.5 ml of DMF, and the mixture is stirred for 0.5 h. 50 μl (0.39 mmol, 0.9 eq.) of triethylamine and 60 μl (0.47 mmol, 1.1 eq.) of 2,2-dimethyl-propanoyl chloride are then added. The reaction mixture is stirred at RT for 1 h, 55 mg (0.43 mmol, 1.0 eq.) of 4-chloroaniline are then added and the mixture is stirred at RT for a further 4 h. After addition of water and saturated aqueous sodium bicarbonate solution and phase separation, the aqueous phase is extracted with ethyl acetate. The combined organic phases are dried over sodium sulphate, filtered and concentrated under reduced pressure. The crude product is purified by preparative HPLC.

Yield: 66 mg (purity 70%, 70% of theory)

LC-MS (method 1): Rₜ=2.80 min;

MS (ESIpos): m/z=611 [M+H]⁺.

### Step b): N-(4-Chlorophenyl)-N′-{4-[2-(2-hydroxyethyl)-3-oxomorpholin-4-yl]phenyl}pyrazine-2,3-dicarboxamide

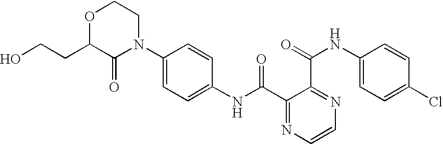

At RT, 160 μl (0.16 mmol, 2.0 eq.) of tetra-n-butylammonium fluoride are added to a solution of 66 mg (70%, 0.08 mmol) of N-{4-[2-(2-{[tert-butyl(dimethyl)silyl]oxy}ethyl)-3-oxomorpholin-4-yl]phenyl}-N′-(4-chlorophenyl)pyrazine-2,3-dicarboxamide in 1 ml of THF, and the mixture is stirred at RT overnight. The reaction mixture is diluted with water and dichloromethane and, after phase separation, the aqueous phase is extracted with dichloromethane. The combined organic phases are dried over magnesium sulphate, filtered and concentrated under reduced pressure.

Yield: 25 mg (purity 97%, 64% of theory)

LC-MS (method 1): Rₜ=1.63 min;

MS (ESIpos): m/z=496 [M+H]⁺,

¹H-NMR (400 MHz, CDCl₃): δ=9.05 (d, 2H), 8.7 (s, 2H), 7.73 (d, 2H), 7.68 (d, 2H), 7.3 (dd, 4H), 4.42 (t, 1H), 4.12-4.2 (m, 1H), 3.98 (d, 2H), 3.89-3.8 (m, 2H), 3.62-3.52 (m, 1H), 2.4 (t, 1H), 2.32-2.08 (m, 2H).

According to the General method 1, the following compounds are prepared starting with Example 19A:

### Example 22

### N-(4-Chlorophenyl)-N′-[4-(3-methyl-2-oxotetrahydropyrimidin-1 (2H)-yl)phenyl]pyrazine-2,3-dicarboxamide

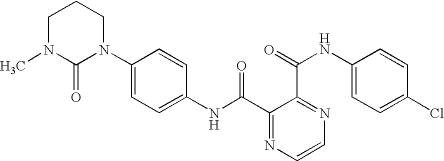

Yield: 17.3 mg (37% of theory)

LC-MS (method 1): Rₜ=1.85 min;

MS (ESIpos): m/z=465 [M+H]⁺,

¹H-NMR (400 MHz, DMSO-d₆): δ=10.85 (s, 1H), 10.69 (s, 1H), 8.94 (s, 2H), 7.8 (d, 2H), 7.66 (d, 2H), 7.42 (d, 2H), 7.21 (d, 2H), 3.62 (t, 2H), 3.39-3.34 (m, 2H), 2.85 (s, 3H), 1.98-2.09 (m, 2H).

### Example 23

### N-(5-Chloropyridin-2-yl)-N′-[4-(3-methyl-2-oxotetrahydropyrimidin-1(2H)-yl)phenyl]pyrazine-2,3-dicarboxarmide

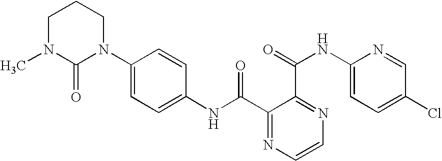

Yield: 2.3 mg (5% of theory)

LC-MS (method 3): Rₜ=1.93 min;

MS (ESIpos): m/z=466 [M+H]⁺.

### Example 24

### N-(6-Chloropyridin-3-yl)-N′-[4-(3-methyl-2-oxotetrahydropyrimidin-1(2H)-yl)phenyl]pyrazine-2,3-dicarboxamide

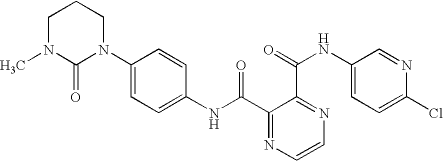

Yield: 9 mg (19% of theory)

LC-MS (method 3): Rₜ=1.74 min;

MS (ESIpos): m/z=466 [M+H]⁺.

### Example 25

### N-(4-Fluorophenyl)-N′-[4-(3-methyl-2-oxotetrahydropyrimidin-1 (2H)-yl)phenyl]pyrazine-2,3-dicarboxamide

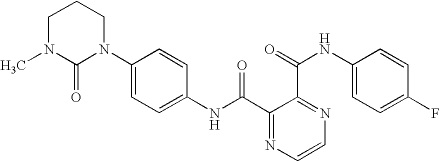

Yield: 30 mg (67% of theory)

LC-MS (method 4): Rₜ=2.07 min;

MS (ESIpos): m/z=448 [M+H]⁺.

### Example 26

### N-[4-(3-methyl-2-oxotetrahydropyrimidin-1(2H)-yl)phenyl]-N′-(4-methylphenyl)pyrazine-2,3-dicarboxamide

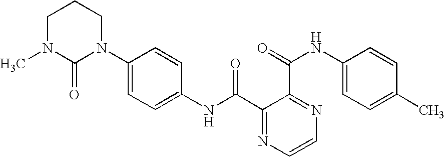

Yield: 22 mg (49% of theory)

LC-MS (method 3): Rₜ=1.94 min;

MS (ESIpos): m/z=445 [M+H]⁺.

### Example 27

### N-(5-Chloropyridin-2-yl)-N′-{4-[3-(2-hydroxyethyl)-2-oxotetrahydropyrimidin-1(2H)-yl]phenyl}-pyrazine-2,3-dicarboxamide

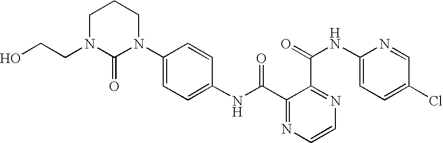

At 0° C., 231 mg (0.61 mmol, 1.1 eq.) of O-(7-azabenzotriazol-1-yl)-N,N,N′,N′-tetramethyluronium hexafluorophosphate (HATU), 130 mg (0.55 mmol) of the compound from Example 10A and 340 μl (1.93 mmol, 3.5 eq.) of N,N-diisopropylethylamine are added to a solution of 169 mg (0.61 mmol, 1.1 eq.) of the compound from Example 12A in 2 ml of dichloromethane and 2 ml of DMF. The reaction mixture is stirred at 0° C. for 30 min and then allowed to warm to RT and stirred overnight. The reaction mixture is concentrated under reduced pressure and the residue is purified by RP-HPLC.

Yield: 17 mg (6% of theory)

LC (method 3): Rₜ=1.74 min;

MS (ESIpos): m/z=496 [M+H]⁺;

¹H-NMR (400 MHz, DMSO-d₆): δ=11.11 (s, 1H), 10.75 (s, 1H), 8.92 (s, 2H), 8.42 (s, 1H), 8.25 (d, 1H), 7.99 (dd, 1H), 7.40 (d, 2H), 7.22 (d, 2H), 4.68 (t, 1H), 3.61 (t, 2H), 3.52 (m, 2H), 3.43 (t, 2H), 2.01 (quintet, 2H).

### Example 28

### N-(5-Chloropyridin-2-yl)-N′-{4-[2-oxo-3-(2-pyrrolidin-1-ylethyl)tetrahydropyrimidin-1(2H)-yl]-phenyl}pyrazine-2,3-dicarboxamide

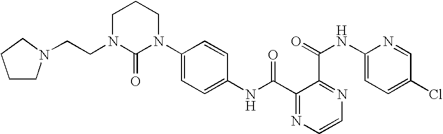

At −78° C., 60 μl (0.34 mmol, 1.2 eq.) of trifluoromethanesulphonic anhydride and 0.10 ml (0.85 mmol, 3 eq.) of 2,6-dimethylpyridine are added to a suspension of 140 mg (0.28 mmol) of the compound from Example 27 in 10 ml of THF and 5 ml of DMF. The reaction mixture is stirred at −78° C. for 2 h and then allowed to slowly warm to −5° C., and 0.24 ml (2.82 mmol, 10 eq.) of pyrrolidine is then added. The reaction solution is slowly warmed to RT and stirred at RT overnight. The reaction mixture is concentrated under reduced pressure and the residue is purified by RP-HPLC.

Yield: 25 mg (15% of theory)

LC (method 3): Rₜ=1.54 min;

MS (ESIpos): m/z=549 [M+H]⁺;

¹H-NMR (300 MHz, DMSO-d₆): δ=11.20-11.03 (br. s, 1H), 10.77 (s, 1H), 8.92 (s, 2H), 8.40 (s, 1H), 8.25 (d, 1H), 8.00 (dd, 1H), 7.70 (d, 2H), 7.23 (d, 2H), 3.65-3.58 (m, 2H), 3.14-3.05 (m, 4H), 2.77-2.60 (m, 6H), 2.07-1.96 (m, 2H), 1.87-1.79 (m, 4H), 1.77-1.68 (m, 4H).

### Example 29

### N-(5-Chloropyridin-2-yl)-N{2-fluoro-4-[3-(hydroxymethyl)-2-oxopyridin-1(2H)-yl]phenyl}pyrazine-2,3-dicarboxamide

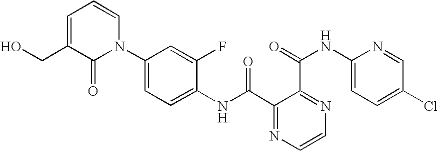

According to the General method 2, 2.00 g (8.54 mmol) of the compound from Example 11A are reacted with 2.38 g (8.54 mmol) of the compound from Example 12A.

Yield: 2.55 g (60% of theory)

LC-MS (method 1): Rₜ=1.58 min;

MS (ESIpos): m/z=495 [M+H]⁺;

¹H-NMR (300 MHz, DMSO-d₆): δ=11.08 (s, 1H), 10.69 (s, 1H), 8.98 (s, 2H), 8.43 (s, 1H), 8.25 (d, 1H), 8.08-7.94 (m, 2H), 7.59 (dd, 1H), 7.51 (dd, 2H), 7.29 (dd, 1H), 6.39 (t, 1H), 5.18 (t, 1H), 4.34 (d, 2H).

### Example 30

### N-(5-Chloropyridin-2-yl)-N′-{4-[3-[(cyclopropylamino)methyl]-2-oxopyridin-1(2H)-yl]-2-fluorophenyl}pyrazine-2,3-dicarboxamide trifluoracetate

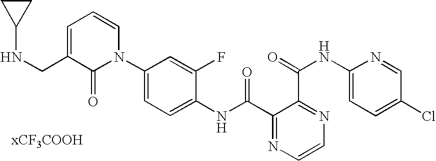

### Step a): N-(5-Chloropyridin-2-yl)-N′-[2-fluoro-4-(3-formyl-2-oxopyridin-1(2H)-yl)phenyl]-pyrazine-2,3-dicarboxamide

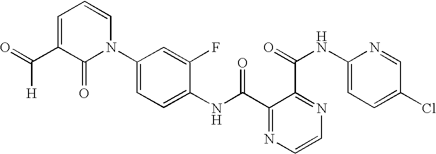

Under argon and at RT, 2.14 g (5.04 mmol, 1.1 eq.) of Dess-Martin periodinane are added to a suspension of 2.27 g (4.58 mmol) of the compound from Example 29 in 60 ml of dichloromethane. The reaction mixture is stirred at RT for 1 h, and within a few minutes the suspension turns into a solution and a precipitate is then formed. The precipitate is filtered off, dried and, without further purification, used for the next step.

Yield: 2.21 g

### Step b): N-(5-Chloropyridin-2-yl)-N′-{4-[3-[(cyclopropylamino)methyl]-2-oxopyridin-1(2H)-yl]-2-fluorophenyl}pyrazine-2,3-dicarboxamide trifluoroacetate

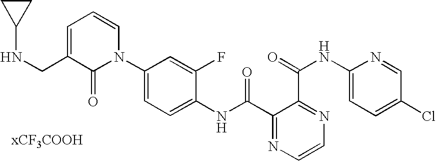

Unter argon and at RT, 70 μl (1.01 mmol, 5 eq.) of cyclopropylamine are added to a suspension of 100 mg (0.20 mmol) of N-(5-chloropyridin-2-yl)-N′-[2-fluoro-4-(3-formyl-2-oxopyridin-1(2H)-yl)phenyl]pyrazine-2,3-dicarboxamide in 5 ml of methanol, and the mixture is stirred at RT for 3 h. The reaction mixture is then cooled in an ice bath, 19 mg (0.51 mmol, 2.5 eq.) of sodium borohydride are added a little at a time and the mixture is then stirred at RT overnight. Saturated aqueous sodium chloride solution and dichloromethane are added to the residue and, after phase separation, the aqueous phase is extracted with dichloromethane. The combined organic phases are dried over magnesium sulphate, filtered and concentrated under reduced pressure. The crude product is purified by preparative HPLC (Kromasil 100 C18 5 μm, mobile phase: water/acetonitrile/1% strength trifluoroacetic acid 48:40:12).

Yield: 4.8 mg (4% of theory)

LC-MS (method 1): Rₜ=1.26 min;

MS (ESIpos): m/z=534 [M+H]⁺;

¹H-NMR (300 MHz, DMSO-d₆): δ=11.08 (s, 1H), 10.70 (s, 1H), 8.98 (d, 2H), 8.75 (br. s, 2H), 8.41 (s, 1H), 8.22 (d, 1H), 8.08 (t, 1H), 7.99 (dd, 1H), 7.86 (dd, 1H), 7.78 (dd, 1H), 7.55 (dd, 1H), 7.32 (dd, 1H), 6.49 (t, 1H), 4.10 (s, 2H), 2.78-2.69 (m, 1H), 0.84-0.75 (m, 4H).

### Example 31

### N-(4-Cyanophenyl)-N′-[4-(3-oxomorpholin-4-yl)phenyl]pyrazine-2,3-dicarboxamide

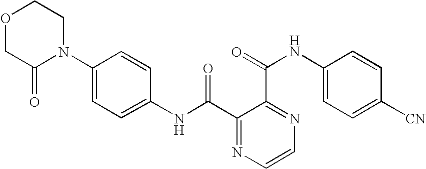

According to the General method 2, 143 mg (0.75 mmol) of the compound from Example 6A are reacted with 200 mg (0.75 mmol) of the compound from Example 21A.

Yield: 24 mg (7% of theory)

LC-MS (method 3): Rₜ=1.63 min;

MS (ESIpos): m/z=443 [M+H]⁺;

¹H-NMR (400 MHz, DMSO-d₆): δ=11.18 (s, 1H), 10.87 (s, 1H), 8.98 (s, 2H), 7.96 (d, 2H), 7.83 (d, 2H), 7.79 (d, 2H), 7.38 (d, 2H), 4.20 (s, 2H), 3.98 (t, 2H), 3.72 (t, 2H).

### Example 32

### N-[2-(2-Aminoethoxy)-4-(3-oxomorpholin-4-yl)phenyl]-N′-(5-chloropyridin-2-yl)pyrazine-2,3-dicarboxamide trifluoroacetate

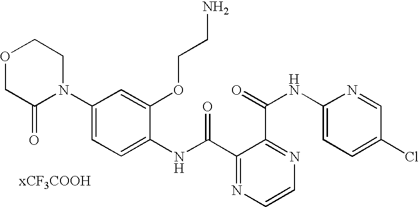

### Step a): N-(5-Chloropyridin-2-yl)-N′-[2-[2-(1,3-dioxo-1,3-dihydro-2H-isoindol-2-yl)ethoxy]4-(3-oxomorpholin-4-yl)phenyl]pyrazine-2,3-dicarboxamide

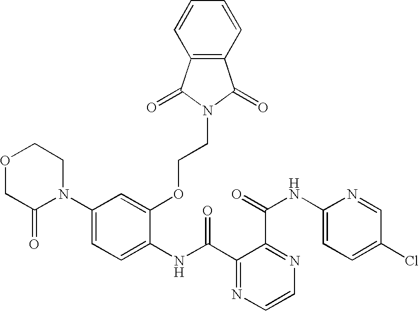

According to the General method 2, 146 mg (0.52 mmol) of the compound from Example 12A are reacted with 200 mg (0.52 mmol, 1.0 eq.) of the compound from Example 22A. The mixture is stirred at room temperature for 16 hours, and another 563 mg (2.02 mmol, 3.9 eq.) of the compound from Example 12A are then added. After a further 16 hours of stirring, 20 ml of water are added and the organic phase is removed. The latter is washed with 20 ml of a saturated aqueous sodium bicarbonate solution and with 20 ml of water. The organic phase is concentrated under reduced pressure and the residue is purified by preparative RP-HPLC.

Yield: 127 mg (38% of theory)

LC-MS (method 3): Rₜ=2.18 min;

MS (ESIpos): m/z=642 [M+H]⁺;

¹H-NMR (400 MHz, DMSO-d₆): δ=11.10 (s, 1H), 10.03 (s, 1H), 8.93-8.91 (m, 1H), 8.79-8.77 (m, 1H), 8.38-8.36 (m, 1H), 8.23 (d, 1H), 8.20 (d, 1H), 7.96 (d, 1H), 7.82-7.75 (m, 4H), 7.24-7.22 (m, 1H), 6.95 (d, 1H), 4.37 (t, 2H), 4.16 (s, 2H), 4.02 (t, 2H), 3.94 (t, 2H), 3.68 (t, 2H).

### Step b): N-[2-(2-Aminoethoxy)-4-(3-oxomorpholin-4-yl)phenyl]-N′(5-chloropyridin-2-yl)-pyrazine-2,3-dicarboxamide trifluoroacetate

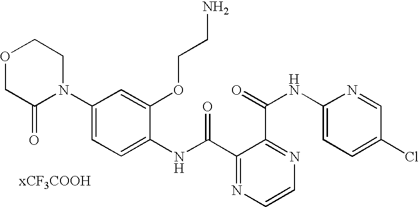

A suspension of 113 mg (0.18 mmol) of N-(5-chloropyridin-2-yl)-N′-[2-[2-(1,3-dioxo-1,3-dihydro-2H-isoindol-2-yl)ethoxy]-4-(3-oxomorpholin-4-yl)phenyl]pyrazine-2,3-dicarboxamide and 0.205 ml (82.0 mg, 2.64 mmol, 15 eq.) of a 40% strength aqueous methylamine solution in 5 ml of methanol is stirred at 40° C. for 15 minutes. The mixture is then stirred at 60° C. for 25 minutes, then cooled to room temperature and concentrated, and the residue is purified by preparative RP-HPLC (mobile phase: water/acetonitrile/1% strength trifluoroacetic acid 56:30:14).

Yield: 61 mg (52% of theory)

LC-MS (method 3): Rₜ=1.33 min;

MS (ESIpos): m/z=512 [M+H—CF₃COOH]⁺;

¹H-NMR (400 MHz, DMSO-d₆): δ=11.16 (s, 1H), 10.26 (s, 1H), 8.99-8.97 (m, 1H), 8.95-8.93 (m, 1H), 8.44 (s, 1H), 8.23 (d, 1H), 8.07 (d, 1H), 8.01 (br. s, 3H), 8.01-7.98 (m, 1H), 7.24 (s, 1H), 7.03 (d, 1H), 4.29 (t, 2H), 4.21 (s, 2H), 3.99 (t, 2H), 3.74 (t, 2H), 3.29-3.27 (m, 2H).

B. Evaluation of the Pharmacological Activity

The compounds according to the invention act in particular as selective inhibitors of blood coagulation factor Xa and do not, or only at significantly higher concentrations, inhibit other serine proteases, such as plasmin or trypsin.

Inhibitors of blood coagulation factor Xa are referred to as being “selective” if the IC₅₀ values for factor Xa inhibition is smaller by a factor of at least 100 compared with the IC₅₀ values for the inhibition of other serine proteases, in particular plasmin and trypsin, where, with a view to the test methods for selectivity, reference is made to the test methods described below of Examples B.a.1) and B.a.2).

The advantageous pharmacological properties of the compounds according to the invention can be determined by the following methods:

a) Test Description (In Vitro)

A.1) Determination of the Factor Xa Inhibition:

The enzymatic inhibition of human factor Xa (FXa) is measured using the conversion of a chromogenic substrate specific for FXa. Factor Xa cleaves p-nitroaniline from the chromogenic substrate. The determinations are carried out in microtitre plates as follows:

The test substances, in various concentrations, are dissolved in DMSO and incubated for 10 minutes at 25° C. with human FXa (0.5 nmol/l dissolved in 50 mmol/l of Tris buffer [C,C,C-tris(hydroxymethyl)aminomethane], 150 mmol/l of NaCl, 0.1% BSA [bovine serum albumin], pH=8.3). Pure DMSO is used as control. The chromogenic substrate (150 μgmol/l Pefachrome® FXa from Pentapharm) is then added. After an incubation time of 20 minutes at 25° C., the extinction at 405 nm is determined. The extinctions of the text mixtures containing the test substance are compared with the control mixtures without test substance, and the IC₅₀ values are calculated from these data.

Representative activity data from this test are shown in Table 1 below:

TABLE 1

| Example No. | IC₅₀ [nM] |
|---|---|
| 9 | 4.4 |
| 11 | 0.49 |
| 12 | 0.16 |
| 17 | 16 |
| 18 | 7.5 |
| 20 | 6.3 |
| 30 | 0.44 |
| 32 | 0.3 |

a.2) Determination of the selectivity:

To assess selective FXa inhibition, the test substances are examined for their inhibition of other human serine proteases such as trypsin and plasmin. To determine the enzymatic activity of trypsin (500 mU/ml) and plasmin (3.2 nmol/l), these enzymes are dissolved in Tris buffer (100 mmol/l, 20 mmol/l CaCl₂, pH=8.0) and incubated with test substance or solvent for 10 minutes. The enzymatic reaction is then started by adding the corresponding specific chromogenic substrates (Chromozym Trypsin® and Chromozym Plasmin®; from Roche Diagnostics) and the extinction at 405 nm is determined after 20 minutes. All determinations are carried out at 37° C. The extinctions of the test mixtures containing test substance are compared with the control samples without test substance, and the IC₅₀ values are calculated from these data.

a.3) Determination of the anticoagulant action:

The anticoagulant action of the test substances is determined in vitro in human and rabbit plasma. To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 2500 g for 10 minutes. The supernatant is pipetted off. The prothrombin time (PT, synonyms: thromboplastin time, quick test) is determined in the presence of varying concentrations of test substance or the corresponding solvent using a commercial test kit (Hemoliance® RecombiPlastin, from Instrumentation Laboratory). The test compounds are incubated with the plasma at 37° C. for 3 minutes. Coagulation is then started by addition of thromboplastin, and the time when coagulation occurs is determined. Concentration of test substance which effects a doubling of the prothrombin time is determined.

b) Determination of the antithrombotic activity (in vivo)

b.1) Arteriovenous shunt model (rabbit):

Fasting rabbits (strain: Esd: NZW) are anaesthetized by intramuscular administration of Rompun/Ketavet solution (5 mg/kg and 40 mg/kg, respectively). Thrombus formation is initiated in an arteriovenous shunt in accordance with the method described by C. N. Berry et al. [Semin. Thromb. Hemost. 1996, 22, 233-241]. To this end, the left jugular vein and the right carotid artery are exposed. The two vessels are connected by an extracorporeal shunt using a vein catheter of a length of 10 cm. In the middle, this catheter is attached to a further polyethylene tube (PE 160, Becton Dickenson) of a length of 4 cm which contains a roughened nylon thread which has been arranged to form a loop, to form a thrombogenic surface. The extracorporeal circulation is maintained for 15 minutes. The shunt is then removed and the nylon thread with the thrombus is weighed immediately. The weight of the nylon thread on its own was determined before the experiment was started. Before extracorporeal circulation is set up, the test substances are administered either intravenously via an ear vein or orally using a pharyngeal tube.

C. Working Examples of Pharmaceutical Compositions

The compounds according to the invention can be converted into pharmaceutical preparations in the following ways:

Tablet:

Composition:

100 mg of the compound according to the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate. Tablet weight 212 mg. Diameter 8 mm, radius of curvature 12 mm.

Production:

The mixture of the compound according to the invention, lactose and starch is granulated with a 5% strength solution (m/m) of the PVP in water. The granules are dried and then mixed with the magnesium stearate for 5 minutes. This mixture is compressed using a conventional tablet press (see above for the dimensions of the tablet). A compressive force of 15 kN is used as a guideline for the compression.

Suspension which can be Administered Orally:

Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

Composition:

500 mg of the compound according to the invention, 2.5 g of polysorbate and 97 g of polyethylene glycol 400.20 g of oral solution correspond to a single dose of 100 mg of the compound according to the invention.

Production:

The compound according to the invention is suspended in the mixture of polyethylene glycol and polysorbate with stirring. Stirring is continued until the compound according to the invention has dissolved completely.

i.v. solution:

The compound according to the invention is, at a concentration below saturation solubility, dissolved in a physiologically acceptable solvent (for example isotonic saline, glucose solution 5% and/or PEG 400 solution 30%). The solution is subjected to sterile filtration and filled into sterile and pyrogen-free injection containers.

1. A compound of the formula (I) 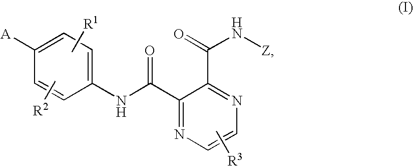
in which
A represents a group of the formula 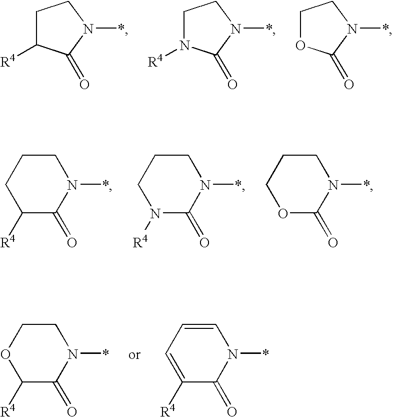
in which
R⁴ represents hydrogen, (C₁-C₆)-alkyl, hydroxyl, (C₁-C₆)-alkoxy, amino, mono- or di-(C₁-C₆)-alkylamino, (C₃-C₇)-cycloalkylamino, (C₁-C₆)-alkanoylamino or (C₁-C₆)-alkoxycarbonylamino, where (C₁-C₆)-alkyl, (C₁-C₆)-alkoxy, mono- and di-(C₁-C₆)-alkylamino for their part may in each case be substituted by hydroxyl, (C₁-C₄)-alkoxy, amino, mono- or di-(C₁-C₄)-alkylamino, (C₃-C₇)-cycloalkylamino or a 4- to 7-membered saturated heterocycle which is attached via a nitrogen atom and which may contain a ring member from the group consisting of N—R⁵ and 0, in which
R⁵ represents hydrogen or (C₁-C₄)-alkyl,
and * represents the point of attachment to the phenyl ring,
z represents phenyl, pyridyl, pyrimidinyl, pyrazinyl or thienyl which may in each case be mono- or disubstituted by identical or different substituents selected from the group consisting of fluorine, chlorine, cyano, (C₁-C₄)-alkyl (which for its part may be substituted by amino) ethynyl and amino,
R¹ and R² are identical or different and independently of one another represent hydrogen, fluorine, chlorine, cyano, (C₁-C₃)-alkyl, cyclopropyl, trifluoromethyl, hydroxyl, (C₁-C₃)-alkoxy, trifluoromethoxy or amino, where (C₁-C₃)-alkyl and (C₁-C₃)-alkoxy for their part may be substituted by hydroxyl or amino,
and
R³ represents hydrogen or (C₁-C₆)-alkyl, which may be substituted by hydroxyl, (C₁-C₄)-alkoxy, amino or mono- or di-(C₁-C₄)-alkylamino,
or pharmaceutically acceptable salt thereof.

2. The compound of claim 1, in which
A represents a group of the formula 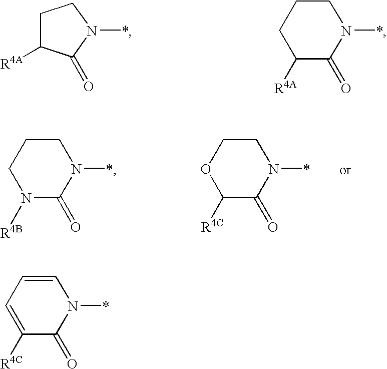
in which
R^{4A} represents hydrogen, hydroxyl, methoxy or amino,
R^{4B} represents methyl or ethyl, each of which may be substituted by hydroxyl, amino, pyrrolidino or cyclopropylamino, or amino,
R^{4c} represents hydrogen, methyl or ethyl, where methyl or ethyl may in each case be substituted by hydroxyl, amino, pyrrolidino or cyclopropylamino, and
represents the point of attachment to the phenyl ring,
z represents a group of the formula 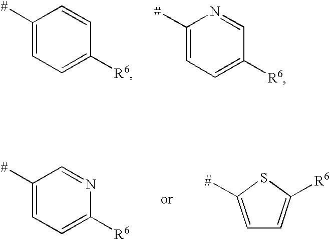
in which
R⁶ represents fluorine, chlorine, methyl, cyano or ethynyl and
# represents the point of attachment to the nitrogen atom,
R¹ represents hydrogen,
R² represents hydrogen, fluorine or methyl,
and
R³ represents hydrogen,
or pharmaceutically acceptable salt thereof.

3. The compound of claim 1, in which
A represents a heterocyclic group of the formula 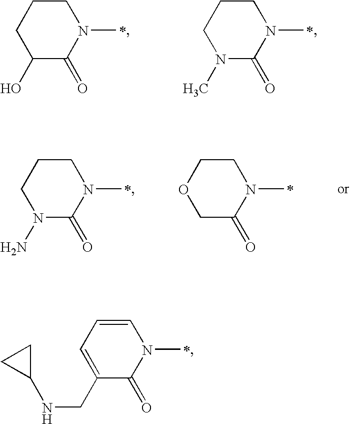
in which * represents the point of attachment to the phenyl ring,
Z represents a group of the formula 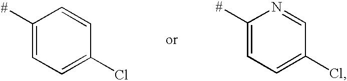
in which # represents the point of attachment to the nitrogen atom,
R¹ represents hydrogen,
R² represents hydrogen, fluorine or methyl,
and
R³ represents hydrogen,
or pharmaceutically acceptable salt thereof.

4. Process for preparing compounds of the formula (I) as defined in claim 1, characterized in that either
[A]compounds of the formula (II) 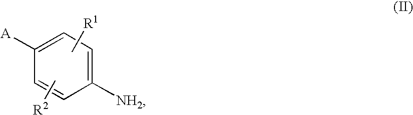
in which A, R¹ and R² are as defined in claim 1
are initially reacted with a compound of the formula (III) 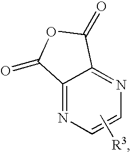
in which R³ is as defined in claim 1
to give compounds of the formula (IV) 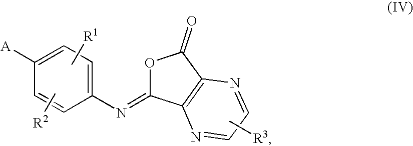
in which A, R¹, R² and R³ are as defined in claim 1
and these are then converted with a compound of the formula (V)
H₂N-Z (V),
in which Z is as defined in claim 1
into compounds of the formula (I)
or
[B] compounds of the formula (V) are initially reacted with a compound of the formula (III) to give compounds of the formula (VI) 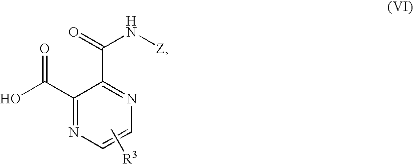
in which R³ and Z are as defined in claim 1
and these are then converted with a compound of the formula (II) into compounds of the formula (I),
and the compounds of the formula (I) are optionally converted with the appropriate bases or acids into their pharmaceutically acceptable salts.

5. (canceled)

6. A method for treating or preventing thromboembolic disorders, comprising administering to a patient a therapeutically effective amount of a compound of claim 1.

7. A method for preventing blood coagulation in vitro, comprising adding an effective amount of a compound of claim 1 to blood.

8. A pharmaceutical composition, comprising a compound of the formula (I) as defined in claim 1 in combination with an inert nontoxic pharmaceutically acceptable auxiliary.

9. The pharmaceutical composition of claim 8, comprising a further active compound.

10. (canceled)

11. A method for treating or preventing thromboembolic disorders in humans or animals which comprises using an anticoagulatory effective amount of at least one compound of the formula (I) as defined in claim 1 or a pharmaceutical composition as defined in claim 8 or 9.

12. The method of claim 7, wherein the effective amount is an anticoagulatory effective amount.

13. The pharmaceutical composition of claim 9, wherein the further active compound is selected from the group consisting of
lipid-lowering agents;
coronary therapeutics/vasodilators;
plasminogen activators (thrombolytics/fibrinolytics) and compounds which increase thrombolysis/fibrinolysis;
substances having anticoagulant activity (anticoagulants);
platelet aggregation-inhibiting substances (platelet aggregation inhibitors); and
fibrinogen receptor antagonists (glycoprotein IIb/IIIa antagonists).

14. The pharmaceutical composition of claim 13, wherein the lipid-lowering agent is a HMG-CoA (3-hydroxy-3-methylglutaryl-coenzym A) reductase inhibitor.

15. The pharmaceutical composition of claim 13, wherein the coronary therapeutic/vasodilator is an ACE (angiotensin converting enzyme) inhibitors; AII (angiotensin II) receptor antagonist; β-adrenoceptor-antagonist; alpha-1-adrenoceptor antagonist; diuretic; calcium channel blocker; substance which bring about an increase in cyclic guanosine monophosphate (cGMP).

16. The pharmaceutical composition of claim 13, wherein the plasminogen activators (thrombolytics/fibrinolytics) and compounds which increase thrombolysis/fibrinolysis are inhibitors of plasminogen activator inhibitor (PAI inhibitors) or inhibitors of thrombin-activated fibrinolysis inhibitor (TAFI).
